(19) United States
(12) Patent Application Publication
(10) Pub. No.: US 2009/0099623 A1
(43) Pub. Date: Apr. 16, 2009

(54) Systems and methods for treatment of medical conditions related to the central nervous system and for enhancing cognitive functions

(75) Inventor: Jonathan Bentwich, Zipori (IL)

Correspondence Address: DICKSTEIN SHAPIRO LLP, 1825 EYE STREET NW, Washington, DC, 20006-5403, US

(73) Assignee: Neuronix Ltd.

(21) Appl. No.: 12/285,416

(22) Filed: Oct. 3, 2008

Related U.S. Application Data
(63) Continuation-in-part of application No. 12/153,037, filed on May 13, 2008.
Application No. 12/153,037 is a continuation of application No. 10/904,505, filed on Nov. 14, 2004, now abandoned.
(60) Provisional application No. 60/960,574, filed on Oct. 4, 2007.
(60) Provisional application No. 60/522,286, filed on Sep. 13, 2004.

(51) Int. Cl. A61N 1/36; A61B 5/0478; A61N 2/00; A61K 35/12

(52) U.S. Cl. 607/45; 600/544; 600/9; 424/93.1

(57) ABSTRACT

Systems and methods for diagnosing and treating various brain-related conditions, and/or for modifying at least one of cognitive, behavioral, or affective functions or skills in individuals. The method of diagnosing and treating a brain-related condition includes the steps of: (i) identifying at least a brain region associated with the brain-related condition; (ii) stimulating the brain region by employing at least one electrical, magnetic, electromagnetic, and photoelectric stimulus; (iii) optionally, stimulating at least one cognitive feature associated with the brain region; and (iv) optionally, subjecting the brain region to a treatment including at least one of cell replacement therapy, cell regenerative therapy and cell growth.

### CROSS-REFERENCE TO RELATED APPLICATIONS

This application claims the benefit of U.S. Provisional Application No. 60/960,574, filed Oct. 4, 2007, entitled “Method and System for Enhancement of Cognitive Functions and Helmet for Treatment of Central Nervous System Medical Implications,” the entire disclosure of which is incorporated by reference in its entirety herein. This application is also a continuation-in-part of U.S. application Ser. No. 12/153,037, filed May 13, 2008, which is a continuation of U.S. application Ser. No. 10/904,505, filed Nov. 14, 2004, which in turn claims the benefit of U.S. Provisional Application No. 60/522,286, filed Sep. 13, 2004, the entire disclosures of which are also incorporated by reference in their entirety herein.

This application is related to Attorney Docket No. N2222.0008/P008, entitled “Systems and Methods for Assessing and Treating Medical Conditions Related to the Central Nervous System and for Enhancing Cognitive Functions,” filed on even day herewith, and incorporated by reference in its entirety herein, which non-provisional application claims the benefit of U.S. Provisional Application No. 60/960,575, filed Oct. 4, 2007, entitled “System and Method for Assessment and Treatment of Central Nervous System Medical Implications and Indications,” the entire disclosure of which is incorporated by reference in its entirety.

### FIELD OF THE INVENTION

The present invention relates to systems and methods for diagnosing and treating medical conditions associated with the neural system, and for enhancing cognitive functions in individuals.

### BRIEF SUMMARY OF THE INVENTION

The present invention provides methods and systems configured to identify and treat various medical conditions associated with the neural system. The present invention also provides systems and devices for enhancing cognitive functions in individuals.

Other features and advantages of the present invention will become apparent from the following description of the invention, which refers to the accompanying drawings.

### BRIEF DESCRIPTION OF THE DRAWINGS

FIG. 1 is a schematic block-diagram of an integrative neuro-cognitive system according to an exemplary embodiment of the present invention;

FIG. 2 is a schematic block-diagram of the NEURODIAGNOSTICS MODULE of the system of FIG. 1;

FIG. 3 is a schematic block-diagram of the REGIONS OF INTEREST COMPUTATIONAL MODULE of the system of FIG. 1;

FIG. 4 is a schematic block-diagram of the BRAIN TRAIT COMPUTATION MODULE of the system of FIG. 1;

FIG. 5 is a schematic block-diagram of the TREATMENT MODULE of the system of FIG. 1;

FIG. 6 is a schematic block-diagram of the STIMULATION MODULE of the system of FIG. 1;

FIG. 7 is a schematic block-diagram of the BRAIN STIMULATOR of the STIMULATION MODULE of FIG. 6;

FIG. 8 is another schematic representation of the BRAIN STIMULATOR of the STIMULATION MODULE of FIG. 6;

FIG. 9 illustrates the system for Embodiment A;

FIG. 10 illustrates the system for Embodiment B;

FIG. 11 illustrates the system for Embodiment C;

FIG. 12 is a computer application block diagram;

FIG. 13 is the END Block Diagram;

FIG. 14 is the ISAT Inter-Subject Across Time Block Diagram;

FIG. 15 is the NDA Normative Data Analysis Block Diagram;

FIG. 16 is the EDMIS Expert Decision Making Interactive System Block Diagram;

FIG. 17 is the ADM Alzheimer's Diagnostic Module Block Diagram;

FIG. 18 is the DBLM Diseased Brain Localization Module Block Diagram;

FIG. 19 illustrates an enhanced version of the stimulator of Embodiment C; and

FIG. 20 illustrates a schematic illustration of the gyroscope stabilization and feedback system of the integrative neuro-cognitive system of the present invention.

### DETAILED DESCRIPTION OF THE INVENTION

The examples provided below detail the various embodiments of the present invention. Other features, embodiments, and advantages of the invention beyond those discussed in the detailed description will become more apparent to those skilled in the art in views of details provided herein. Those skilled in the art should appreciate that many changes may be made to the present invention without departing from the scope or spirit of the present invention.

The present invention provides methods and systems configured to identify and treat various medical conditions associated with the neural system. The present invention also provides methods and systems for enhancing cognitive functions in individuals.

The present invention provides systems and apparatus configured to identify and treat various brain-related conditions and/or to assess and modify (for example, enhance) at least one of cognitive, behavioral, or affective function or skill in individuals. The system may include at least one stimulator. A suitable stimulator includes, but is not limited to, a first stimulator, which may include at least one of invasive and non-invasive brain stimulation devices, and a second stimulator which is operatively connected to the first stimulator. The first stimulator is configured to stimulate at least one brain region associated with a brain-related condition by employing at least one of electrical, magnetic, electromagnetic, and photoelectric stimuli. The second stimulator is configured to modify at least one cognitive function associated with the identified brain region. The first and second stimulators may form a single integrated device or, alternatively, may form separate parts of the device. The first and second stimulators are configured to operate simultaneously or sequentially.

The present invention also provides methods of diagnosing and treating various brain-related conditions and/or of modifying at least one cognitive, behavioral, or affective function or skill in individuals. The method of diagnosing and treating a brain-related condition or for enhancing a cognitive function may include the steps of: (i) identifying at least a brain region associated with the brain-related condition or the cognitive function; (ii) stimulating the brain region by employing a stimulus such as electrical, magnetic, electromagnetic, and photoelectric stimuli; (iii) optionally, stimulating at least one cognitive feature associated with the brain region of at least step (i); (iv) optionally, subjecting the brain region of at least step (i) to a treatment involving at least one of cell replacement therapy, cell regenerative therapy and cell growth; and (v) optionally, subjecting the brain region of at least step (i) to a pharmacological treatment.

The present invention provides integrative neuro-cognitive systems for diagnosing and treating various brain-related diseases, and/or for assessing and enhancing particular cognitive, behavioral, or affective functions (or skills) in brain-related cognitive functions in normal individuals (based on an individual-based comparison of structural or functional or cognitive functioning with corresponding statistical health or brain diseases norms or with statistical norms for cognitively enhanced functions). The integrative neuro-cognitive system of the present invention also provides subsequent neuronal electrical or electromagnetic stimulation, and convergent cognitive stimulation of the identified diseased brain regions in an individual or sub-enhanced cognitive function or functions of brain regions.

The invention also provides neurodiagnostic computational systems and methodology for differentially diagnosing an individual with a particular brain-related disease or diseases, along with a specification of the individual's particular functional, structural, or cognitive abnormalities. Alternatively, the invention provides neurodiagnostic computational systems and methodology for identifying those particular cognitive function or functions, which may be further enhanced in an individual relative to cognitively enhanced standards for brain functions. Additionally, the invention also provides apparatus and methods of computing a precise individual-based brain stimulation, and corresponding cognitive stimulation parameters, needed to stimulate the identified disease-related brain loci, or to enhance an identified cognitive skill or function.

The invention further provides apparatus and methods for stimulating the relevant brain regions and corresponding cognitive functions, while continuously monitoring and adjusting the brain and cognitive stimulation parameters for a given individual or a disease or a particular cognitive enhancement function, based on a comparison of pre- and post-stimulation neurodiagnostic measurements of the relevant brain function, structure, and corresponding cognitive functions.

The invention provides methodology and system for precisely locating and stimulating electrically or electromagnetically the relevant diseased brain regions or regions whose stimulation may improve cognitive performance in a particular skill or skills in normal individuals. The electrical or electromagnetic stimulation may be combined with convergent cognitive stimulation of the same brain regions, and/or with in-vivo regenerative or neuronal implantation of neuroplasticity methodologies which trigger a regeneration, replacement, or growth of the same brain regions stimulated electrically or electromagnetically or cognitively, to maximize the potential therapeutic or neuroplasticity effect, or with any pharmaceutical agent or material which may facilitate the neuroplasticity or regenerative or enhancement of cognitive functions associated with the same brain region or regions being stimulated electromagnetically or cognitively etc.

The present invention also describes a computerized statistical assessment methodology and systems for differentiating between individuals with enhanced cognitive function or functions and normal individuals.

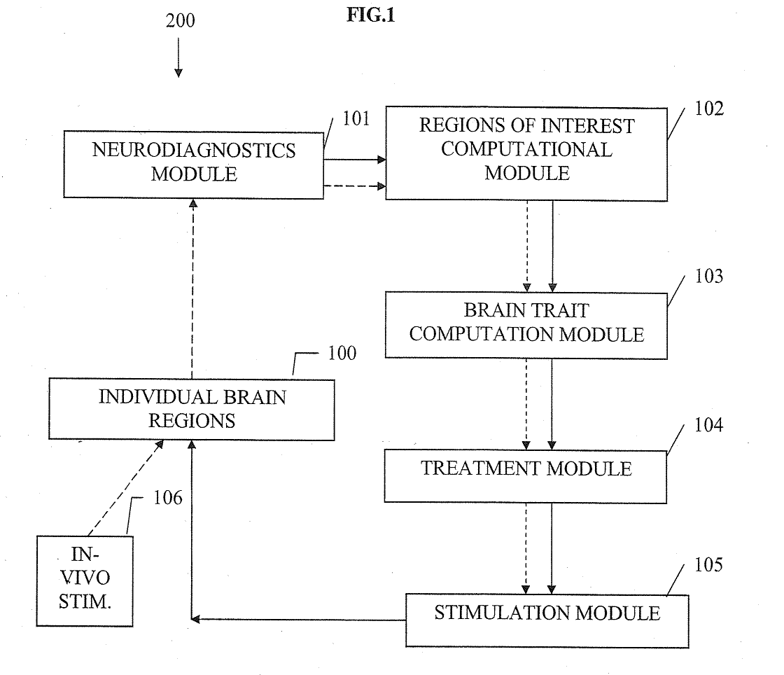

Referring now to the drawings, where like elements are designated by like reference numerals, FIGS. 1-8 illustrate various structural elements of system 200 of the present invention configured to diagnose and treat medical conditions associated with the neural system, and/or to enhance cognitive functions in mammals.

Reference is made to FIG. 1, which illustrates INDIVIDUAL BRAIN REGIONS 100 that are pathological functional or structural brain features, or cognitive performance features in an individual, which are associated with a specific brain-related disease that is identified by a NEURODIAGNOSTICS MODULE 101 (FIG. 1). NEURODIAGNOSTICS MODULE 101 measures the functional activation or structural maps, or corresponding cognitive performance in an individual for a particular task (or tasks) or during a resting period. NEURODIAGNOSTICS MODULE 101 transfers this information to REGIONS OF INTEREST COMPUTATIONAL MODULE 102, which identifies those particular brain regions in an individual whose structure, function, or cognitive functions are deviant from their corresponding statistically-established health norms, or from their corresponding statistical norms for cognitively enhanced performance in a particular task.

REGIONS OF INTEREST COMPUTATIONAL MODULE 102 outputs these identified statistically-deviant or cognitively-enhanced brain regions in a given individual for analysis in a BRAIN TRAIT COMPUTATION MODULE 103. The BRAIN TRAIT COMPUTATION MODULE 103 determines whether or not any of these identified brain regions statistically fits within known structural, functional, or cognitive pathophysiology of a particular brain-related disease. Alternatively, BRAIN TRAIT COMPUTATION MODULE 103 determines whether or not any of these identified brain regions statistically fits within established norms for enhanced or excellent cognitive or behavioral performance (in a particular task or skill or skills). Thus, for example, in the case of Autism Spectrum Disorder (ASD), statistically-established norms indicate that autistic children or individuals exhibit an abnormal deficient activation (as well as structurally decreased size) of the left hemisphere's (LH) typical Broca's and Wernicke's language regions, while abnormally hyperactivating (or structurally enlarged) contralateral (RH) Broca's and Wernicke's regions. Therefore, in cases in which the REGIONS OF INTEREST COMPUTATIONAL MODULE 102 identifies such abnormal hypoactivation of the LH's Broca's and Wernicke's language regions (with or without an accompanying hyperactivation of the contralateral RH's Broca's and Wernicke's regions), the COMPUTATIONAL MODULE 102 then outputs these regions to the BRAIN TRAIT COMPUTATION MODULE 103, to determine whether or not any of these identified brain regions statistically fits within known structural, functional, or cognitive pathophysiology of Autism Spectrum Disorder (ASD).

Alternatively, in the case of Alzheimer's disease (or any other memory loss that is due to aging, dementia or mild cognitive impairment (MCI)), statistically established norms indicate that such memory impairment is associated with decreased structure and function of the hippocampus and other medial temporal structures, as well as decreased connectivity between frontal and posterior brain regions and facial recognition regions, or structural, functional, or cognitive impairment of the cerebellum (associated with impaired motor coordination and semantic memory or verbal capability loss), or impairment of mood and executive functioning regions (such as the left prefrontal region and cingulate gyrus and frontal lobe). Therefore, in cases in which the REGIONS OF INTEREST COMPUTATIONAL MODULE 102 identifies such abnormally-decreased structural or functional values of these brain structures, these brain regions are output to the BRAIN TRAIT COMPUTATION MODULE 103, to determine whether or not any of these identified brain regions statistically fits within known structural, functional, or cognitive pathophysiology of Alzheimer's, MCI, dementia, or age-related memory loss, or other aging illnesses. In those cases in which the identified regions of interest or cognitive performance levels match the brain disease, or match the neural functional, structural, or cognitive levels of a sub-cognitively enhanced performance in a particular task or tasks, the TREATMENT MODULE 104 computes the precise individual-based brain and cognitive stimulation parameters needed to stimulate the identified INDIVIDUAL BRAIN REGIONS 100 that are necessary to improve the functional, structural or cognitive disease indices, or to enhance performance in a particular task or tasks.

The REGIONS OF INTEREST COMPUTATIONAL MODULE 102 also outputs identified cognitively enhanced brain regions in a given individual for analysis in the BRAIN TRAIT COMPUTATION MODULE 103, to determine whether or not any of these identified brain regions statistically deviates from the established norms for enhanced or excellent cognitive or behavioral performance (in a particular task or skill or skills). Thus, for instance, in the case of a normal individual whose structural, functional or cognitive patterns are found to be statistically different than the norms for enhanced language capabilities which are indicated by above-normal or exceptional cognitive language capabilities including but not limited to naming, articulation, short-term verbal memory, measures of verbal intelligence, word association, vocabulary, syntax, pragmatic language, semantics etc., which are also associated with enhanced functional activation or connectivity or efficient brain activation patterns or any other measure of functional or structural enhanced cognitive language performance capabilities, then cognitive or electromagnetic or electrical stimulation of these identified sub-enhanced brain regions or corresponding cognitive functions will be performed. In those cases in which the identified regions of interest or cognitive performance levels are computed to match sub-enhanced neural functioning or structural or cognitive correlates in a particular task or tasks the TREATMENT MODULE 104 computes the precise individual-based brain and cognitive stimulation parameters needed in order to improve the functional, structural or cognitive geared towards enhancing performance in a particular task or tasks.

The STIMULATION MODULE 105 receives input from the TREATMENT MODULE 104 regarding an individual-based brain and cognitive stimulation including their integrated neuro-cognitive stimulation parameters. Additionally and/or optionally, an IN-VIVO STIMULATOR 109 may be combined with the STIMULATION MODULE 105. In an exemplary embodiment, IN-VIVO STIMULATOR 109 may include in-vivo transplantation or regenerative or stem-cell insertion of neuronal cells or tissue or supportive cells targeting the same INDIVIDUAL BRAIN REGIONS 100.

A feedback may be also combined with the STIMULATION MODULE 105, and following the STIMULATION MODULE 105. The feedback may include a post-stimulation measurement carried out by the NEURODIAGNOSTICS MODULE 101 which then undergoes all the sequential computational steps including: the REGIONS OF INTEREST COMPUTATIONAL MODULE 102, the BRAIN TRAIT COMPUTATION MODULE 103, the TREATMENT MODULE 104, and the STIMULATION MODULE 105. All feedback computational steps are aimed towards monitoring and adjusting the individual-based brain and corresponding cognitive stimulation parameters continuously, based on the potential improvement in functional, structural, or corresponding cognitive stimulation in an individual following the administration of brain stimulation and corresponding cognitive stimulation (e.g., until a certain pathophysiological disease threshold has been transcended indicating clinical improvement in that individual or, alternatively, until a certain cognitive enhancement threshold has been transcended indicating an enhancement of a particular cognitive function or functions in an individual).

Each of the components of FIG. 1 (i.e., the NEURODIAGNOSTICS MODULE 101, the REGIONS OF INTEREST COMPUTATIONAL MODULE 102, the BRAIN TRAIT COMPUTATION MODULE 103, the TREATMENT MODULE 104, and the STIMULATION MODULE 105) can function independently or separately, or in any possible combination with each other.

In accordance with one embodiment of the present invention, the NEURODIAGNOSTICS MODULE 101 is configured to translate functional or structural neuroimaging data into statistically valid individual functional activation patterns and statistically valid individual structural maps. The NEURODIAGNOSTICS MODULE 101 is also configured to compare individual cognitive performance data with statistically established health norms.

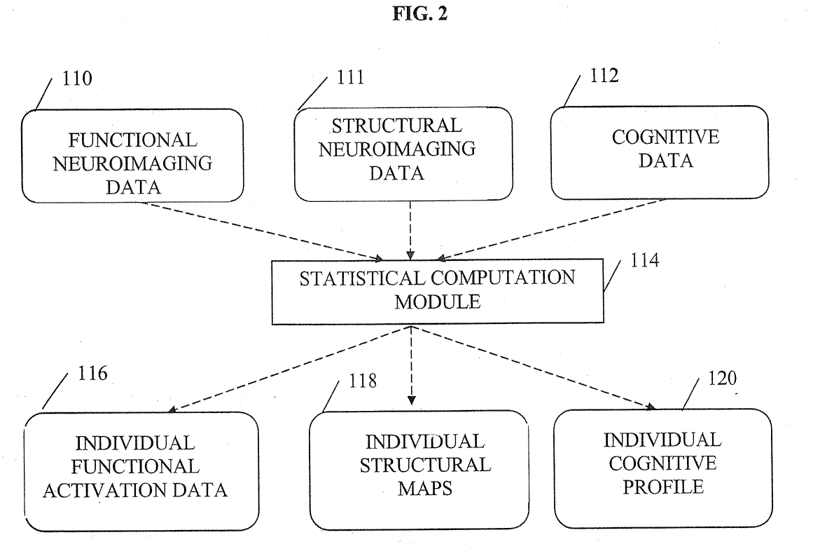

Reference is now made to FIG. 2, which illustrates a simplified block diagram of the NEURODIAGNOSTICS MODULE 101 of system 200 of FIG. 1. NEURODIAGNOSTICS MODULE 101 is configured to obtain a FUNCTIONAL NEUROIMAGING DATA 110, a STRUCTURAL NEUROIMAGING DATA 111, and a COGNITIVE DATA 112, that are then fed into a STATISTICAL COMPUTATION MODULE 114. As shown in FIG. 2, STATISTICAL COMPUTATION MODULE 114 is configured to compute an INDIVIDUAL FUNCTIONAL ACTIVATION DATA 116, an INDIVIDUAL STRUCTURAL MAPS 118, and an INDIVIDUAL COGNITIVE PROFILE 120.

The FUNCTIONAL NEUROIMAGING DATA 110 includes various neuroimaging measurements of activation across different brain regions of a specific individual, during the performance of a particular cognitive or behavioral task. Another possible measurement of the FUNCTIONAL NEUROIMAGING DATA 110 includes neuroimaging measurements of a specific individual while at rest. This data can be obtained through the use of various magnetic resonance imagining (MRI), functional magnetic resonance imagining (fMRI), positron emission tomography (PET), single photon emission computerized tomography (SPECT), electroencephalography (EEG) and event related potentials (ERP) techniques, among many others.

The STRUCTURAL NEUROIMAGING DATA 110 includes various neuroimaging measurements of an individual's brain structure. A non-limiting example of structural mapping is the MRI (although, as detailed above, other devices such as PET and SPECT are also capable of generating structural images).

The COGNITIVE DATA 112 includes measurements of cognitive performance of an individual in a wide range of possible cognitive or behavioral tests, which may include but are not limited to: response times, accuracy, measures of attention, memory, learning, executive function, language, intelligence, personality measures, mood, and self-esteem, among others. The cognitive data may be obtained through computerized, paper and pencil, interviewing, performance tests or other forms of administering the cognitive or behavioral tests. The cognitive data may be obtained via verbal, written, visual or tactile responses which are input into the computer in various forms.

As shown in FIG. 2, the FUNCTIONAL NEUROIMAGING DATA 110, the STRUCTURAL IMAGING DATA 111, and the COGNITIVE DATA 112 are input into the STATISTICAL COMPUTATION MODULE 114 which compares each of these types of data to statistically established norms, to determine an INDIVIDUAL FUNCTIONAL ACTIVATION DATA 116, an INDIVIDUAL STRUCTURAL MAPS 118, and an INDIVIDUAL COGNITIVE PROFILE 120. Various computational softwares for performing those computational and analyses are available, such as ICA, SPM and AutoROI, among many others.

Based on the analysis of the STATISTICAL COMPUTATION MODULE 114 of the individual's functional patterns relative to the statistically established norms, the INDIVIDUAL FUNCTIONAL ACTIVATION DATA 116 provides unique brain activation patterns of an individual performing a specific cognitive or behavioral task, or while resting, relative to a statistically established norm.

Similarly, based on the analysis of the STATISTICAL COMPUTATION MODULE 114 of the individual's structural brain images relative to statistically established norms, the INDIVIDUAL STRUCTURAL MAPS 118 provides unique brain structure of an individual.

Based on the analysis of the STATISTICAL COMPUTATION MODULE 114 of the individual's cognitive performance relative to statistically established norms, the INDIVIDUAL COGNITIVE PROFILE 120 includes that individual's unique cognitive capabilities, skills or functions.

The NEURODIAGNOSTICS MODULE 101 may consist of the FUNCTIONAL NEUROIMAGING DATA 110, the STRUCTURAL NEUROIMAGING DATA 111, the COGNITIVE DATA 112, together or separately, or in any combination. However, the STATISTICAL COMPUTATION MODULE 114 is a part of the NEURODIAGNOSTICS MODULE 101 in any combination.

A constraint imposed on the possible combinations of these components is that, if the FUNCTIONAL NEUROIMAGING DATA 110 inherently exists in the individual, then the INDIVIDUAL FUNCTIONAL ACTIVATION DATA 116 must exist; if the STRUCTURAL NEUROIMAGING DATA 111 inherently exists in the individual, then the INDIVIDUAL STRUCTURAL MAPS 118 must exist; and, if the COGNITIVE DATA 112 inherently exists in the individual, then the INDIVIDUAL COGNITIVE PROFILE 120 must exist.

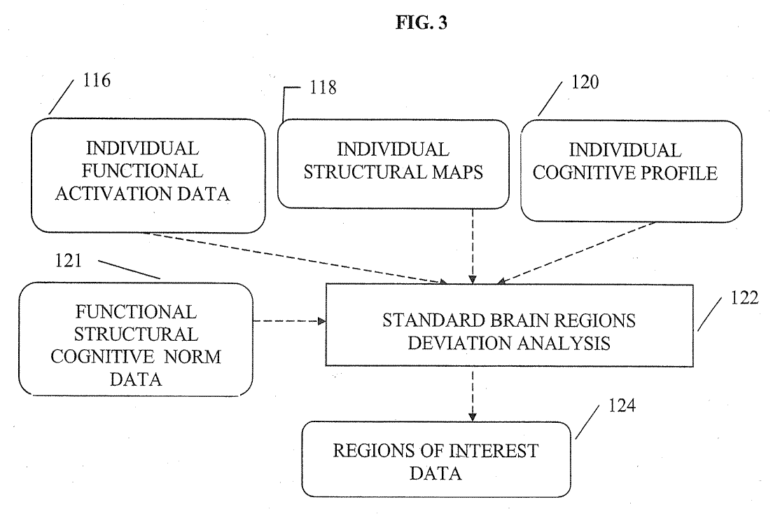

Reference is now made to FIG. 3, which is a simplified illustration of the REGIONS OF INTEREST COMPUTATION MODULE 102 of system 200 of FIG. 1. The REGIONS OF INTEREST COMPUTATION MODULE 102 is configured to identify a disease-specific and individual-specific pathophysiological brain regions. Alternatively, the REGIONS OF INTEREST COMPUTATION MODULE 102 is configured to identify the particular functional or structural brain loci, or corresponding cognitive characteristics, that are different in a given normal individual from their corresponding attributes in statistical standard of excellence or enhanced performance in a particular cognitive skill or function associated with a particular brain region.

Input from the INDIVIDUAL FUNCTIONAL ACTIVATION DATA 116, the INDIVIDUAL STRUCTURAL MAPS 118, and the INDIVIDUAL COGNITIVE PROFILE 120 of FIG. 2, and a FUNCTIONAL, STRUCTURAL, COGNITIVE NORM DATA 121 are received by the STANDARD BRAIN REGIONS DEVIATION ANALYSIS 122, which determines which brain regions exhibit a deviation from statistically established health norms in terms of functional activation patterns, structure, or corresponding cognitive performance levels and is output as the REGIONS OF INTEREST DATA 124. Alternatively, the STANDARD BRAIN REGIONS DEVIATION ANALYSIS 122 is configured to determine which brain regions exhibit a deviation from a statistical established norm for functional activation patterns, brain structure, and cognitive features of a particular excellent or enhanced cognitive or behavioral performance that is output as the REGIONS OF INTEREST DATA 124.

Each of the three INDIVIDUAL FUNCTIONAL ACTIVATION DATA 116, INDIVIDUAL STRUCTURAL MAPS 118, and INDIVIDUAL COGNITIVE PROFILE 120 can function independently or separately, or in any possible combination with the other two modules. However, at least one of these three modules must accompany the FUNCTIONAL, STRUCTURAL, COGNITIVE NORM DATA 121 and the STANDARD BRAIN REGIONS DEVIATION ANALYSIS 122, to compute and output the REGIONS OF INTEREST DATA 124 (which are the particular functional, structural, or corresponding cognitive brain regions which exhibit statistically deviant values relative to the distribution of the normal population or, alternatively, relative to the distribution of enhanced cognitive performance corresponding to functional, structural, or cognitive performance levels).

In accordance with one embodiment of the present invention, the STANDARD BRAIN REGIONS DEVIATION ANALYSIS 122 relies on statistical computation which compares an individual's functional activation patterns to statistically established health norms (which may rely on known standards of normal brain activation during the performance of a particular cognitive or behavioral task or tasks or at rest, or it may rely on a statistical comparison of the individual to a sufficiently large sample of functional activation patterns in a group of normal matched controls performing a particular cognitive-behavioral task or tasks). The comparison of the individual's functional activation patterns, brain structure, or cognitive performance to statistically established health norms relies on a statistical contrast between the individual's cognitive performance values (pixel by pixel, or region by region, functional and structural, or particular brain regions) with the corresponding values of a normally distributed healthy control group or population.

The goal of any one of a variety of statistical procedures known in the art is to determine the likelihood of the individual's functional, structural or cognitive values (parsed by cell, region, brain structure, lobe or hemisphere levels) as belonging to the normal distribution of corresponding functional, structural, or cognitive values in normal controls. Different confidence intervals, significance thresholds, and means of reducing error rate etc. can be utilized to determine those regions of interest which are different in the individual relative to the control group.

In accordance with another embodiment of the present invention, the STANDARD BRAIN REGIONS DEVIATION ANALYSIS 122 may rely on statistical computation which compares an individual's functional activation patterns to statistically established norms for excellent or enhanced particular cognitive, or behavioral performance, in above-average individuals, or following enhancing brain stimulation of the regions corresponding to a particular cognitive function, or enhancing cognitive training of the same particular cognitive function or skill. The comparison of the individual's functional activation patterns, brain structure or cognitive performance to statistically-established norms of functional, structural, or cognitive performance in individuals who exhibit excellent cognitive performance in a particular task or skill can rely on a statistical contrast of the individual's pixel by pixel, or region by region, functional and structural or cognitive performance values with the corresponding values of a normally-distributed healthy control group or population. The goal of any one of a variety of statistical procedures known in the art is to determine the likelihood of the individual's functional, structural, or cognitive values (parsed by cell, region, brain structure, lobe or hemisphere levels) as belonging to the (normal) distribution of corresponding functional, structural, or cognitive values in excellent or enhanced cognitive performance in a particular task or skill from individual normal controls, or following a cognitive training of that particular function, or through enhancing that cognitive function through stimulation of the corresponding brain regions.

The STANDARD BRAIN REGIONS DEVIATION ANALYSIS 122 outputs the REGIONS OF INTEREST DATA 124, the particular structural brain loci, functional brain regions, and cognitive features that are deviant from the statistically established functional or structural brain norms. Alternatively, the STANDARD BRAIN REGIONS DEVIATION ANALYSIS 122 outputs the REGIONS OF INTEREST DATA 124 that may includes the particular structural brain loci, functional brain regions, and cognitive features that are different from the statistically established functional or structural brain norms for a standard of a particular excellent or enhanced cognitive performance.

Several examples for possible REGIONS OF INTEREST DATA 124 in the case of an individual at risk for developing (or already exhibiting) abnormal functional, structural or corresponding cognitive performance abnormalities associated with Alzheimer disease are as follows: abnormally deficient activation of left frontal, left prefrontal, Broca's, Wernicke's, hippocampus and related regions, anterior cingulated, and also motor, medial temporal gyrus, anthreonal gyrus, cerebellum, and a decline in functional connectivity measures between some or all of these regions. Structural abnormalities may also consist of a decrease in these structures volume or connecting fibers between these neuronal regions. In the case of autism spectrum disorder (ASD), structural abnormalities are evidenced by reversed functional activation of right hemisphere RH instead of left hemisphere LH language regions activation patterns in ASD children (and adults) relative to normal matched controls, e.g., hypoactivation of LH's Broca's, Wernicke's regions but hyperactivation of these contralateral regions in the RH in the ASD relative to matched controls. For “Theory of Mind” social cognition ASD deficits, functional hypoactivation of the Amygdala, fusiform gyrus, and dysfunction of inter-hemispheric connectivity measures may occur. Additionally, a generalized RH dysfunction in the ASD individuals relative to controls which may manifest as a generalized RH hyperactivation in Theory of Mind paradigms, at resting conditions or in language paradigms, may occur.

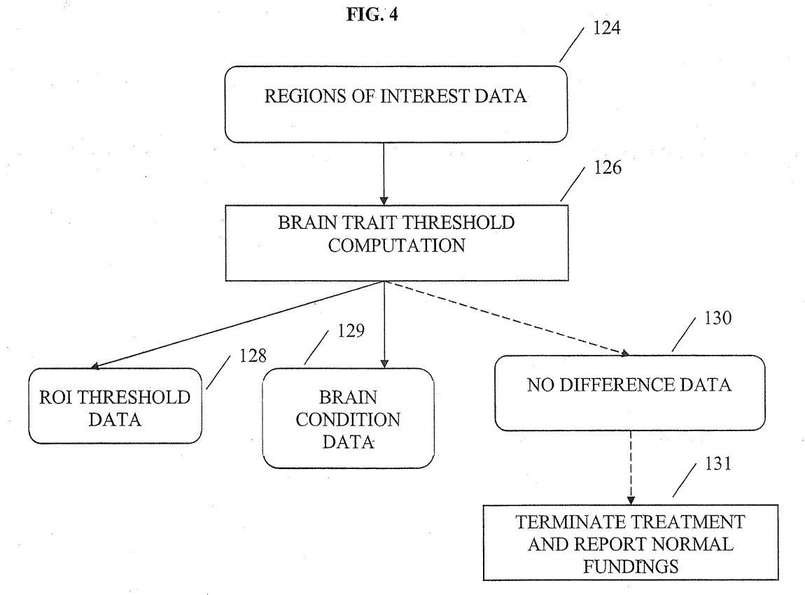

Reference is now made to FIG. 4, which depicts the BRAIN TRAIT COMPUTATION MODULE 103 of system 200 of FIG. 1. BRAIN TRAIT COMPUTATION MODULE 103 is configured to determine whether or not the identified REGIONS OF INTEREST DATA 124 signify a likelihood of the individual being afflicted by a specific functional, structural, or corresponding cognitive impairment related to a specific brain-related disease. Alternatively, the BRAIN TRAIT COMPUTATION MODULE 103 of FIG. 1 is configured to determine whether or not the identified REGIONS OF INTEREST DATA 124 signify the likelihood of an individual being below enhanced or excellent functional, structural, or corresponding cognitive-task performance criteria (e.g., in terms of functional, structural, or cognitive values relative to their corresponding values in a sample of individuals with excelled performance).

The REGIONS OF INTEREST DATA 124 (which are those brain regions for which the functional activation, structure, or corresponding cognitive performance has been determined to be statically different in an individual than in the control group or, alternatively, relative to a sample of cognitively enhanced performance) is input into the BRAIN TRAIT THRESHOLD COMPUTATION 126. The BRAIN TRAIT THRESHOLD COMPUTATION 126 determines which of these REGIONS OF INTEREST DATA 124 has a functional activation, or structural properties, or corresponding cognitive performance values that are different from disease-specific statistical threshold values that have a high predictive value for the existence of a specific disease in an individual at the time of testing or prospectively at different time points. Alternatively, the REGIONS OF INTEREST DATA 124 is input into the BRAIN TRAIT THRESHOLD COMPUTATION 126 which determines whether these REGIONS OF INTEREST DATA 124 have functional activation or structural values that are the same as, or different from, the statistically determined functional or structural values threshold for a particularly enhanced cognitive function or functions.

In cases in which the BRAIN TRAIT THRESHOLD COMPUTATION 126 determines that the REGIONS OF INTEREST (ROI) DATA 124 are same as, or exceed, the threshold for functional or structural values of a particular region or regions that have been determined as characterizing a particular disease, then it will output an ROI THRESHOLD DATA 128 and a BRAIN CONDITION DATA 129. For those functional, structural, or corresponding cognitive performance threshold values of a particular brain-related disease which are characterized as being below the statistically computed values of the normal control population, then, if an individual's REGIONS OF INTEREST DATA 124 are below the above-mentioned disease-specific threshold, the BRAIN TRAIT THRESHOLD COMPUTATION 126 will output the ROI THRESHOLD DATA 128 as consisting of all the REGIONS OF INTEREST DATA 124 that are below-threshold regions for a particular brain-related disease specified by the BRAIN CONDITION DATA 129. In those cases in which the BRAIN TRAIT THRESHOLD COMPUTATION 126 detects statistically significant functional, structural, or corresponding cognitive performance values in an individual that exceed the disease-specific threshold values or, alternatively, are below the disease-specific threshold in cases in which the functional, structural, or corresponding cognitive performance values have been determined to be statistically below those of normal controls, the BRAIN TRAIT THRESHOLD COMPUTATION 126 will also output a BRAIN CONDITION DATA 129 with a specification of what particular brain-related disease is statistically reliably associated with these above-threshold (or below-threshold as explained above) functional, structural, or corresponding cognitive performance values in a given individual.

In cases in which the functional, structural, or corresponding cognitive performance values in an individual have not exceeded the disease-specific threshold (or in cases in which the disease-specific threshold is below the statistical values in the normal population and the individual's ROI THRESHOLD DATA 128 is above these disease-specific thresholds), then the BRAIN TRAIT THRESHOLD COMPUTATION 126 will output a NO DIFFERENCE DATA 130 (e.g., indicating that no functional, structural, or cognitive patterns exist in the individual that are different from the statistical distribution of normal individuals). In this case, the NO DIFFERENCE DATA 130 instigates a TERMINATE TREATMENT AND REPORT NORMAL FINDINGS 131, which terminates the diagnostic phase of the invention with an output to the individual, or the treating clinician, that the individual is not likely to suffer from any brain-related disease and, therefore, no treatment is warranted.

In cases in which the BRAIN TRAIT THRESHOLD COMPUTATION 126 determines that the REGIONS OF INTEREST DATA 124 are same as, or exceed, the threshold for functional or structural values of a particular region or regions that have been determined as characterizing an enhanced performance or function in a particular cognitive task or skill, then it will output an ROI THRESHOLD DATA 128 and a BRAIN CONDITION DATA 129. For those functional or structural values that are associated with a particularly enhanced cognitive skill or function which are characterized as being below the statistically computed values of the normal control population, then, if an individual's REGIONS OF INTEREST DATA 124 are below the above-mentioned cognitive enhanced threshold, the BRAIN TRAIT THRESHOLD COMPUTATION 126 will output the ROI THRESHOLD DATA 128 consisting of all the REGIONS OF INTEREST DATA 124 that are below-threshold regions. In those cases in which the BRAIN TRAIT THRESHOLD COMPUTATION 126 detects statistically significant functional or structural values in an individual that exceed the cognitively enhanced threshold values or, alternatively, are below the cognitive enhanced threshold values in cases in which the functional or structural values have been determined to be statistically below those of normal controls, the BRAIN TRAIT THRESHOLD COMPUTATION 126 also outputs a BRAIN CONDITION DATA 129 which includes a specification of what particular cognitively enhanced skills or functions are statistically reliably associated with these above-threshold (or below-threshold as explained above) functional, structural, or corresponding cognitive performance values in a given individual.

In those cases in which the BRAIN TRAIT THRESHOLD COMPUTATION 126 outputs the ROI THRESHOLD DATA 128 and BRAIN CONDITION DATA 129, the ROI THRESHOLD DATA 128 includes the identification of all the pixels, or cellular, or regional, or hemispheric brain regions for which the functional, structural, or corresponding cognitive performance levels in an individual have been computed to exceed the disease-specific threshold in an individual or be below the disease-specific threshold (as shown above), and an indication of the precise functional or structural or cognitive values of each of these pixels, or cellular or regional or hemispheric loci relative to their corresponding disease-specific threshold. In those cases in which the BRAIN TRAIT THRESHOLD COMPUTATION 126 outputs the ROI THRESHOLD DATA 128 and BRAIN CONDITION DATA 129, and in which the ROI THRESHOLD DATA 128 includes the identification of all pixels, or cellular, or regional, or hemispheric brain regions for which the functional, structural, or corresponding cognitive performance levels in an individual have been computed to be lower than the enhanced cognitive performance level in a particular cognitive task or function (or be below the particularly enhanced cognitive threshold as shown above), the ROI THRESHOLD DATA 128 also specifies the precise functional, structural, or cognitive values at each of the identified pixels, cellular or regional or hemispheric loci—along with their corresponding statistically computed thresholds.

In cases in which the functional, structural, or corresponding cognitive performance values in an individual have not exceeded the disease-specific threshold (or in cases in which the disease-specific threshold is below the statistical values in the normal population and the individual's ROI THRESHOLD DATA 128 is above these disease-specific thresholds), then the BRAIN TRAIT THRESHOLD COMPUTATION 126 outputs a NO DIFFERENCE DATA 130 (e.g., indicating that no functional, structural, or cognitive patterns exist in the individual that are different from the statistical distribution of normal individuals). In this case, the NO DIFFERENCE DATA 130 instigates a TERMINATE TREATMENT AND REPORT NORMAL FINDINGS 131, which terminates the diagnostic phase of the invention with an output to the individual or the treating clinician that the individual is not likely to suffer from any brain-related disease and, therefore, that no treatment is warranted.

In cases in which the functional or structural values in an individual have not exceeded the cognitively-enhanced threshold (or in cases in which the cognitively enhanced threshold is below the statistical values in the normal population and the individual's ROI THRESHOLD DATA 128 is above these particular cognitively enhanced threshold), then the BRAIN TRAIT THRESHOLD COMPUTATION 126 outputs a NO DIFFERENCE DATA 130 (e.g., indicating that no functional, structural, or cognitive patterns exist in the individual that are different from the statistical distribution of cognitively enhanced functional or structural features). In this case, the NO DIFFERENCE DATA 130 instigates a TERMINATE TREATMENT AND REPORT NORMAL FINDINGS 131, which terminates the diagnostic phase of the invention with an output to the individual or the treating clinician that the individual is not likely to benefit from any cognitive enhancement treatment.

The computation carried out by the BRAIN TRAIT THRESHOLD COMPUTATION 126 is based upon a statistical comparison of an individual's functional activation, brain structure, or cognitive performance with a statistical distribution of the corresponding functional, structural, or cognitive performance in particular brain-related diseases. Alternatively, the computation carried out by the BRAIN TRAIT THRESHOLD COMPUTATION 126 may be based upon a statistical comparison of an individual's functional activation, brain structure, or cognitive performance with a statistical distribution of the corresponding functional, structural, or cognitive performance for particularly enhanced cognitive skills or functions. These statistical comparisons consist of a pixel by pixel, cellular, regional, or hemispheric comparison of that individual's REGIONS OF INTEREST DATA 124 with its corresponding statistical norms for specific diseases or, alternatively, for particularly enhanced cognitive functions. These statistical norms for normal functional, structural, or corresponding cognitive performance may be obtained through meta-analysis (or other statistical procedures) for averaging scientifically published data quantifying functional, structural, or corresponding cognitive performance levels at different pixel, cellular, regional or hemispheric levels, and across different neuroimaging paradigms in a specific disease and a particular sub-phenotype or stage of the specific disease.

Alternatively, these statistically computed norms for normal brain functioning, structure, and corresponding cognitive performance may be obtained through a sufficiently large sample of normal vs. diseased individuals for a specific disease, with subsequent statistical methods being utilized to normalize the distribution of normal controls vs. diseased individuals which would result in the computation of a specific statistical threshold for each pixel, cell, region or hemisphere—above or below which values in an individual are likely to represent a specific disease, sub-phenotype or stage of a particular disease. Alternatively, these statistically computed norms for normal brain functioning, structure and corresponding cognitive performance can be obtained through a sufficiently large sample size of normal vs. enhanced cognitive skill or skills performance individuals for a specific skill with subsequent statistical methods being utilized to normalize the distribution of normal controls vs. enhanced cognitive performance individuals which would result in the computation of a specific statistical threshold for each pixel, cell, region or hemisphere—above or below which values in an individual are likely to represent a specific enhanced cognitive performance or skill or skills. Moreover, varying the significance level, confidence interval, power of test, effect size or other statistical measures which quantify the difference between a particular brain diseased population and normal control population based on a sample from these populations—may allow one to obtain different statistical (predictive) thresholds for distinguishing a brain-related disease from normal control values.

The BRAIN TRAIT THRESHOLD COMPUTATION 126 determination of the statistical threshold above- or below-which functional, structural, or corresponding cognitive performance levels are likely to represent a particular brain disease, sub-phenotype, or disease-stage depends upon the analysis of the normal vs. diseased sample distribution (i.e., in those cases in which the statistical analysis has demonstrated that the normal sample yields statistically reliable higher functional or structural values for a particular pixel, cell, region, or hemisphere than the disease sample, then the BRAIN TRAIT THRESHOLD COMPUTATION 126 will determine that values in an individual for that particular pixel, cell, region hemisphere etc. which are below the computed threshold for normal population values will be marked as a diseased region for a particular disease). Thus for example, statistical analyses have demonstrated that the normal sample yields statistically reliable higher functional or structural values for the LH's Broca's and Wernicke's regions than values for an autism sample. Therefore, the BRAIN TRAIT THRESHOLD COMPUTATION 126 will determine that an individual who exhibits functional activation, structural volume, or cognitive values for those particular brain regions which are below the computed threshold for the corresponding normal population values will be marked as a diseased region for autism, in that particular individual. Similarly, statistical analyses have demonstrated that the normal sample yields statistically reliable higher functional activation, structural volume, or cognitive values for the hippocampus, medial temporal structures, connectivity between frontal and posterior or facial recognition or cerebellum or cingulated values than for an Alzheimer's or MCI or demented or aging sample. Therefore, the BRAIN TRAIT THRESHOLD COMPUTATION 126 will determine that an individual who exhibits functional, structural, or cognitive values for those particular brain regions which are below the computed threshold for the corresponding normal population values will be marked as a diseased region for Alzheimer's or MCI or aging diseases.

Conversely, in those cases in which the statistical analysis has demonstrated that the normal sample yields statistically reliable lower functional or structural values for a particular pixel, cell, region, or hemisphere than the disease sample, then the BRAIN TRAIT THRESHOLD COMPUTATION 126 will determine that values in an individual for that particular pixel, cell, region hemisphere etc. which are above the computed threshold for normal population values will be marked as a diseased region for a particular disease. Thus, for example, statistical analyses have shown that the normal sample yields statistically reliable lower functional activation, or structural volume values for the RH's contralateral Broca's or Wernicke's regions than in a sample of autistic children. Therefore, the BRAIN TRAIT THRESHOLD COMPUTATION 126 will determine that values in an individual for the RH's contralateral Broca's or Wernicke's regions that are above the corresponding computed threshold for normal population values will be marked as a diseased region for autism spectrum disorder.

Similarly, in order for the BRAIN TRAIT THRESHOLD COMPUTATION 126 to compute the threshold for functional, structural, or corresponding values indicative of an enhanced cognitive performance in an individual at a particular task or tasks, a statistical comparison of normal vs. enhanced samples or populations will be performed for pixel by pixel, cellular, regional or hemispheric functional, structural or corresponding cognitive measures. In those cases in which the statistical analysis has demonstrated that the enhanced sample yields statistically reliable higher functional or structural values for a particular pixel, cell, region, or hemisphere than in the normal sample or population, the BRAIN TRAIT THRESHOLD COMPUTATION 126 will determine that values in an individual for that particular pixel, cell, region hemisphere etc. which are below the computed threshold for the enhanced population or sample will de determined as indicating that these cellular, regional, or hemispheric regions are indicative of sub-enhanced functional, structural, or corresponding cognitive performance levels in that particular individual. As such, an excitatory stimulation of these identified sub-enhanced brain regions in an individual may enhance their corresponding cognitive performance.

Conversely, in those cases in which the statistical analysis has demonstrated that the enhanced sample yields statistically reliable lower functional or structural values for a particular pixel, cell, region, or hemisphere than the normal sample or population, then the BRAIN TRAIT THRESHOLD COMPUTATION 126 will determine that values that are above the enhanced sample or population's threshold in an individual may indicate a sub-enhanced functional, structural, or corresponding cognitive level in an individual for a particular cognitive trait, performance or skill. As such, inhibitory stimulation of these identified sub-enhanced brain regions in an individual may enhance their corresponding cognitive performance.

The BRAIN TRAIT THRESHOLD COMPUTATION 126 determines whether or not the functional, structural, or corresponding cognitive performance levels in an individual are statistically the “same” or “different” in a given individual relative to their corresponding values in a normal population. Once the BRAIN TRAIT THRESHOLD COMPUTATION 126 has determined that particular REGIONS OF INTEREST DATA 124 do exceed the disease-specific statistical threshold or, alternatively, are below a particular enhanced performance threshold, then it outputs the BRAIN TRAIT DATA 127, which indicates which brain regions are abnormal functionally, structurally, or in terms of their association with particularly impaired cognitive performance, or alternatively which brain regions may be stimulated neuronally or cognitively to enhance a particular cognitive function or skill.

The BRAIN TRAIT THRESHOLD COMPUTATION 126 also outputs the BRAIN THRESHOLD DATA 128, which includes a pixel by pixel, cellular, brain region, or hemispheric values and cognitive performance thresholds for normal brain functioning or, alternatively, for enhanced brain functioning along with various statistical indices associated with these computational thresholds such as significance level, confidence intervals etc., or any other statistical measure that assesses the statistical difference between the REGIONS OF INTEREST DATA 124 functional, structural, or cognitive values and the statistically-established threshold for normal brain functioning. If, on the other hand, the BRAIN TRAIT THRESHOLD COMPUTATION determines that all of the REGIONS OF INTEREST DATA 124 do not exceed the disease-specific statistical threshold or, alternatively, are not below the particular enhanced cognitive performance threshold, then BRAIN TRAIT THRESHOLD COMPUTATION 126 outputs a NO DIFFERENCE DATA 129, which then leads to a TERMINATE TREATMENT AND REPORT NORMAL FINDINGS 130 (which terminates the operation of the medical device and notifies the patient or clinician that the individual is normal with no apparent brain-related disease or, alternatively, performs excellent a particular cognitive task and, therefore, cannot benefit from brain and cognitive stimulation geared towards enhancing particular cognitive skills).

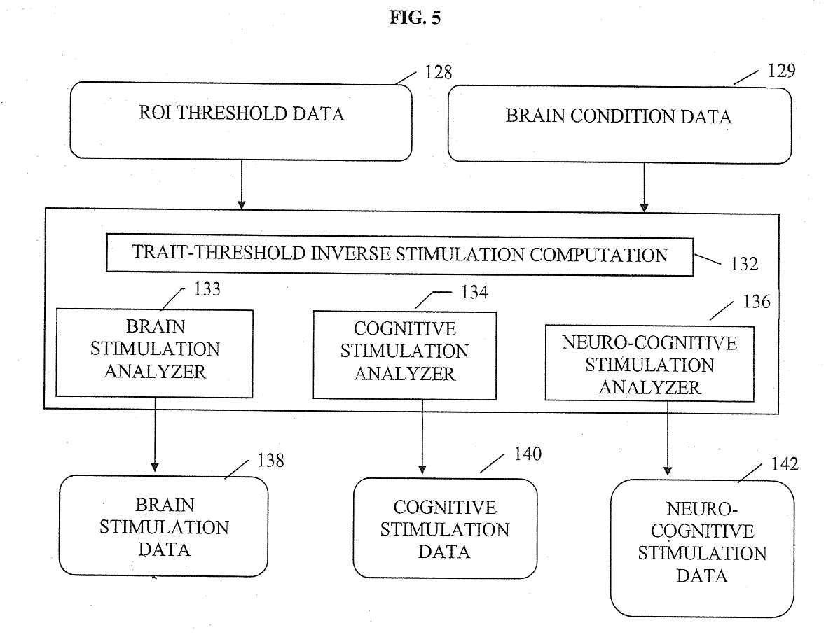

Reference is now made to FIG. 5, which illustrates the TREATMENT MODULE 104 of the system 200 of FIG. 1. The TREATMENT MODULE 104 is configured to determine the precise brain stimulation, cognitive stimulation, and neuro-cognitive stimulation parameters for an individual with a specific brain-related disease. Alternatively, the TREATMENT MODULE 104 is capable of determining the precise brain stimulation, cognitive stimulation and neuro-cognitive stimulation parameters for a normal individual to enhance a particular cognitive function.

The TREATMENT MODULE 104 includes the ROI THRESHOLD DATA 128 and the BRAIN CONDITION DATA 129 of FIG. 4, which are input into a TRAIT-THRESHOLD INVERSE STIMULATION COMPUTATION 132 that includes a BRAIN STIMULATION ANALYZER 133, a COGNITIVE STIMULATION ANALYZER 134, and a NEURO-COGNITIVE STIMULATION ANALYZER 136, which in turn produce a corresponding BRAIN STIMULATION DATA 138, a COGNITIVE STIMULATION DATA 140, and a NEURO-COGNITIVE STIMULATION DATA 140.

The TRAIT-THRESHOLD INVERSE STIMULATION COMPUTATION 132 is configured to compare between the ROI THRESHOLD DATA 128 functional, structural, or cognitive performance levels that are above or below disease-specific thresholds, or are above or below enhanced cognitive performance levels in an individual and their corresponding functional, structural, or corresponding cognitive performance thresholds, and the BRAIN CONDITION DATA 129, to determine the optimal brain, cognitive, or neuro-cognitive stimulation parameters.

A key computational principle guiding the function of the TRAIT-THRESHOLD INVERSE STIMULATION COMPUTATION 132 is that, to improve the functional, structural, or corresponding cognitive performance level in an individual suffering from a particular brain-related disease or, alternatively, to enhance the functional, structural, or corresponding cognitive performance level in a normal individual, it is necessary to stimulate the particularly identified ROI THRESHOLD DATA 128 regions in the inverse excitatory or inhibitory stimulation direction relative to the below or above threshold levels in a given individual. In this manner, in those cases in which an individual's functional, structural, or corresponding cognitive performance levels are below the threshold for corresponding normal functional, structural, or cognitive performance, then the TRAIT-THRESHOLD INVERSE STIMULATION COMPUTATION 132 will compute a generally excitatory brain or cognitive stimulation. For example, in those cases in which an individual's functional, structural, or corresponding cognitive performance levels have been characterized as belonging to autism spectrum disorder's hypoactivation (or abnormally small structure volume) of the LH's Broca's and Wernicke's language regions or of the Aygdala or fusiform gyrus which are below the threshold for corresponding normal functional, structural, or cognitive performance, then the TRAIT-THRESHOLD INVERSE STIMULATION COMPUTATION 132 will compute a generally excitatory brain or cognitive stimulation of these brain regions. Likewise, in those cases in which an individual's functional, structural, or corresponding cognitive performance levels have been characterized as belonging to Alzheimer's, aging, dementia, or MCI which is detected through a hypoactivation (or abnormally small structure volume) of the hippocampus, medial-temporal structures, impairment in connectivity between frontal and posterior or facial recognition regions, or cerebellum or cingulate function or structure, then the TRAIT-THRESHOLD INVERSE STIMULATION COMPUTATION 132 will compute a generally excitatory brain or cognitive stimulation of these brain regions.

Conversely, in those cases in which an individual's functional, structural or corresponding cognitive performance levels are above the threshold for corresponding normal functional, structural, or cognitive performance levels, then the TRAIT-THRESHOLD INVERSE STIMULATION COMPUTATION 132 will compute a generally inhibitory brain or cognitive stimulation. For example, in those cases in which an individual's functional, structural, or corresponding cognitive performance levels have been characterized as belonging to autism spectrum disorder characterized by a hypoactivation (or abnormally small structure volume) of the RH's contralateral Broca's and Wernicke's regions, then the TRAIT-THRESHOLD INVERSE STIMULATION COMPUTATION 132 will compute a generally excitatory brain or cognitive stimulation of these brain regions.

The same trait-threshold inverse stimulation principle also applies to the TRAIT-THRESHOLD INVERSE STIMULATION COMPUTATION 132 for cognitive enhancement. Specifically, in those cases in which an individual's functional, structural or corresponding cognitive performance levels are below the enhanced-cognitive performance threshold, then the TRAIT-THRESHOLD INVERSE STIMULATION COMPUTATION 132 will compute a generally excitatory brain or cognitive stimulation. Conversely, in those cases in which an individual's functional, structural or corresponding cognitive performance levels are above the cognitive-enhancement threshold, then the TRAIT-THRESHOLD INVERSE STIMULATION COMPUTATION 132 will compute a generally inhibitory brain or cognitive stimulation.

In those cases in which an individual's functional, structural or corresponding cognitive performance levels are above the threshold for corresponding enhanced functional, structural or cognitive performance (i.e., such as hyperactivation of a certain brain region that is associated with normal cognitive performance as opposed to a decreased activation of that particular brain region or regions in individuals with enhanced cognitive performance in a particular skill or function or functions), then the TRAIT-THRESHOLD INVERSE STIMULATION COMPUTATION 132 will compute a generally inhibitory brain or cognitive stimulation. Hence, the trait-threshold inverse stimulation principle also applies to the TRAIT-THRESHOLD INVERSE STIMULATION COMPUTATION 132 for cognitive enhancement, namely: in those cases in which an individual's functional, structural or corresponding cognitive performance levels are below the enhanced-cognitive performance threshold, then the TRAIT-THRESHOLD INVERSE STIMULATION COMPUTATION 132 will compute a generally excitatory brain or cognitive stimulation. Conversely, in those cases in which an individual's functional, structural or corresponding cognitive performance levels are above the cognitive-enhancement threshold then the TRAIT-THRESHOLD INVERSE STIMULATION COMPUTATION 132 will compute a generally inhibitory brain or cognitive stimulation.

Specifically, the BRAIN STIMULATION ANALYZER 133 compares between ROI THRESHOLD DATA 128 functional levels that are above or below disease-specific thresholds, or are above or below particular cognitive enhancement thresholds, in a given individual and their corresponding functional threshold, while taking into consideration the BRAIN CONDITION DATA 129 particular brain-related disease, or the particular cognitive enhancement goal—to determine the optimal brain stimulation parameters in a given individual. For example, in cases in which an individual's functional or structural activation parameters are below the normal threshold in certain ROI THRESHOLD DATA 128 regions, then the BRAIN STIMULATION ANALYZER 133 will output excitatory brain stimulation parameters. Conversely, in cases in which an individual's functional or structural activation parameters are above the normal threshold in certain ROI THRESHOLD DATA 128 regions, then the BRAIN STIMULATION ANALYZER 133 will output inhibitory BRAIN STIMULATION DATA 138 parameters.

Similarly, the COGNITIVE STIMULATION ANALYZER 134 compares between ROI THRESHOLD DATA 128 cognitive levels that are above or below disease-specific thresholds, or are above or below particular cognitive enhancement thresholds, in a given individual and their corresponding cognitive thresholds, while taking into consideration the BRAIN CONDITION DATA 129 particular brain-related disease or diseases, or the particular cognitive enhancement goal—to determine the optimal cognitive stimulation parameters in a given individual. For example, in cases in which an individual's cognitive performance level is below the normal threshold for a particular task or function, then the COGNITIVE STIMULATION ANALYZER 133 will output an excitatory cognitive stimulation parameters. Conversely, in cases in which an individual's cognitive performance levels in a particular cognitive function are above the normal threshold, then the COGNITIVE STIMULATION ANALYZER 133 will output inhibitory COGNITIVE STIMULATION DATA 142 parameters (i.e., cognitive stimulation paradigm or training methodology which attempts to inhibit the abnormal (or sub-enhanced) cognitive function either directly or through the training or stimulation of its opposite or complimentary or other cognitive function, which in effect suppresses or diminishes the particular abnormal or sub-enhanced cognitive function).

Likewise, the NEURO-COGNITIVE STIMULATION ANALYZER 136 compares between ROI THRESHOLD DATA 128 functional, structural, or corresponding cognitive performance levels that are above or below disease-specific thresholds, or are above or below particular cognitive enhancement thresholds in a given individual and their corresponding functional threshold, while taking into consideration the BRAIN CONDITION DATA 129 of a particular brain-related disease, or the particular cognitive enhancement goal—in order to determine the optimal brain stimulation parameters in a given individual. However, in the case of the NEURO-COGNITIVE STIMULATION ANALYZER 136, the computation is geared towards identifying the optimal neuro-cognitive stimulation parameters (e.g., in terms of the correspondence between stimulating a specific brain region (or regions) in an excitatory or inhibitory manner and its corresponding cognitive stimulation of the same brain region (or regions) in an inhibitory or excitatory manner, the temporal overlap or separation between the neuronal brain stimulation, and cognitive stimulation of the same or different brain regions, etc.). Thus, based on the ROI THRESHOLD DATA 128 indication of which particular brain region (or regions) is above or below the disease specific or cognitively-enhanced threshold, and which BRAIN CONDITION DATA 129 disease does such above or below threshold individual levels belong to, the NEURO-COGNITIVE STIMULATION ANALYZER 136 computes the above-mentioned optimal neuro-cognitive stimulation parameters.

The specific intensity, duration, loci, interval, and other parameters of brain stimulation computed by the BRAIN STIMULATION ANALYZER 133 are determined based on the input from the BRAIN CONDITION DATA 129 in conjunction with the above-mentioned trait-threshold inverse stimulation principle (e.g., in cases in which the individual's ROI THRESHOLD DATA 128 functional or structural levels are relatively far from the BRAIN CONDITION DATA 129 disease threshold or cognitive enhancement threshold, then the inhibitory or excitatory stimulation parameters would tend to be of higher intensity, duration, multiple brain loci etc., and vice versa).

In order to enhance various cognitive functions or skills the corresponding brain regions should be stimulated excitatorily, i.e., hippocampus or temporal lobe or cingulated gyrus for memory or learning enhancement, frontal or prefrontal cortex for executive functions, concentration, learning, intelligence; motor cortex or cerebellum for motor functions and coordination, visual cortex for enhancing visual functions, inhibitive amygdale for fear and anxiety reduction with or without left frontal and prefrontal excitatory stimulation; enhancement of self-esteem or mood or well-being-excitatory stimulation of left prefrontal or frontal, or inhibitive stimulation of the right prefrontal gyrus. In all these instances corresponding cognitive stimulation can be applied, e.g., which improves or enhances the diseased brain related or cognitive function or enhances the desired cognitive function or functions.

An exemplary embodiment of the present invention encompasses the TREATMENT MODULE 104's tentative ROI THRESHOLD DATA 128 of particular brain-related diseases such as Alzheimer's and ASD's BRAIN CONDITION DATA 129. Specifically, in the case of Alzheimer's, the ROI THRESHOLD DATA 128 is expected to include any one of these regions or any combination thereof: abnormally deficient activation of left frontal, left prefrontal, Broca's, Wernicke's, hippocampus and related regions, anterior cingulated, and also motor, medial temporal gyrus, anthreonal gyrus, cerebellum, and a decline in functional connectivity measures between some or all of these regions. Structural abnormalities may also exist as a decrease in these structures' volume or connecting fibers between these neuronal regions.

In the case of autism spectrum disorder (ASD), ROI THRESHOLD DATA 128 is expected to include any one of these regions or any combination thereof: reversed functional activation of right hemisphere RH instead of left hemisphere LH language regions activation patterns in ASD children (and adults) relative to normal matched controls (e.g., hypoactivation of LH's Broca's, Wernicke's regions but hyperactivation of these contralateral regions in the RH in the ASD relative to matched controls). For “Theory of Mind” social cognition ASD deficits, functional hypoactivation of the Amygdala, fusiform gyrus, and dysfunction of inter-hemispheric connectivity measures may occur. Additionally, a generalized RH dysfunction in the ASD individuals relative to controls which may manifest as a generalized RH hyperactivation in Theory of Mind paradigms, at resting conditions or in language paradigms, may occur.

Accordingly, an exemplary and only illustrative embodiment of the system of the present invention includes BRAIN STIMULATION DATA 138, or COGNITIVE STIMULATION DATA 142, or NEURO-COGNITIVE STIMULATION DATA 140 excitatory stimulation of the left frontal or left prefrontal or Broca's or Wernicke's or hippocampus and related regions or anterior cingulate or motor or medial temporal gyrus, or anthreonal gyrus or cerebellum, or the functional connectivity between some or all of these regions or stimulation of any combination of these regions—in the case of Alzheimer's disease. Likewise, milder cases of Mild Cognitive Impairment (or any other form of age-related memory loss or dementia) may call for similar stimulation of some or all of these brain regions. In the case of ASD, an exemplary embodiment of the system of the present invention may include BRAIN STIMULATION DATA 138, or COGNITIVE STIMULATION DATA 140, or NEURO-COGNITIVE STIMULATION DATA 140 excitatory stimulation of any one of these regions or any combination thereof: Broca's or Wernicke's regions, or Amygdala or fusiform gyrus or of inter-hemispheric connections. Additionally, ASD may call for the BRAIN STIMULATION DATA 138, or COGNITIVE STIMULATION DATA 140, or NEURO-COGNITIVE STIMULATION DATA 140 inhibitory stimulation of the contralateral Broca's or Wernicke's RH regions or a generalized inhibitory stimulation of the RH.

To enhance various cognitive functions or skills, the corresponding brain regions should be stimulated excitatorily, i.e., hippocampus or temporal lobe or cingulated gyrus for memory or learning enhancement, frontal or prefrontal cortex for executive functions, concentration, learning, intelligence; motor cortex or cerebellum for motor functions and coordination, visual cortex for enhancing visual functions, inhibitive amygdale for fear and anxiety reduction with or without left frontal and prefrontal excitatory stimulation; Enhancement of self-esteem or mood or well-being-excitatory stimulation of left prefrontal or frontal, or inhibitive stimulation of the right prefrontal gyrus. In all these cases, corresponding cognitive stimulation may be applied (e.g., stimulus which improves or enhances the disease brain-related or cognitive function or enhances the desired cognitive function or functions).

An important aspect of the TRAIT-THRESHOLD STIMULATION COMPUTATION 132 is the principle of disease-specific or cognitive enhancement specific neuroplasticity computation, which underlies the computation carried out by the NEURO-COGNITIVE STIMULATION ANALYZER 136. This principle embodies the adaptation of various neuro-cognitive stimulation parameters to a specific brain disease, or particular cognitive enhancement protocol, based on the identification of the specific neuroplasticity features that are associated with these particular brain disease or diseases, and cognitive enhancement protocol or protocols. Thus, the NEURO-COGNITIVE STIMULATION ANALYZER 136 takes into account the specific BRAIN CONDITION DATA 129 brain disease or cognitive enhancement goal in a particular individual and, based on this information in conjunction with known neuroplasticity information regarding these ROI THRESHOLD DATA 128 and BRAIN CONDITION DATA 129, the ROI THRESHOLD DATA 128 determines the optimal NEURO-COGNITIVE STIMULATION DATA 140.

The neuroplasticity stimulation parameters may include, for example, the following: the intensity of the brain and corresponding cognitive stimulation, their duration, onset and termination times, temporal overlap or separation, order and combination of all possible brain stimulation loci and their corresponding cognitive stimulations, among others. These parameters may all be dynamically changed or adjusted based on the post-stimulation NEURODIAGNSOTICS MODULE 100 and REGIONS OF INTEREST COMPUTATIONAL MODULE 102 and BRAIN TRAIT COMPUTATION MODULE 103 and TREATMENT MODULE 105.

One example of such NEURO-COGNITIVE STIMULATION ANALYZER 136 is the computation of the optimal neuroplasticity stimulation for treating Alzheimer's memory loss or other MCI, dementia, memory loss diseases, or memory enhancement diseases, which may include: excitatory 10-20 Hz TMS stimulation of the hippocampus or other temporal lobe regions or frontal or prefrontal regions or cingulate gyrus in any possible combination, which will be synchronized with memory enhancement or encoding or retrieval or recall or recognition or mnemonic or perceptual or auditory or semantic memory enhancement cognitive training or stimulation methodologies, to obtain the optimal neuroplasticity potential changes related to memory improvement (e.g., based on the computation of the best neuroplsticity parameters that allow for the most learning, encoding memory retrieval or formation pertaining to these particular ROI THRESHOLD DATA 128 and BRAIN CONDITION DATA 129).

The determination by the NEURO-COGNITIVE STIMULATION ANALYZER 136 of the optimal neuroplasticity parameters specific for a particular ROI THRESHOLD DATA 128 and BRAIN CONDITION DATA 129 may be derived from prior art findings regarding any particular combination of ROI THRESHOLD DATA 128 and BRAIN CONDITION DATA 129. Alternatively, it can be computed based on the present invention's post-stimulation dynamic feedback loop with the above-mentioned NEURODIAGNOSTICS MODULE 100, REGIONS OF INTEREST COMPUTATIONAL MODULE 102, BRAIN TRAIT COMPUTATION MODULE 103, TREATMENT MODULE 105 and STIMULATION MODULE 105. The latter feedback loop computation can allow computation of the most effective learning curve or NEURO-COGNITIVE STIMULATION DATA 140 for a particular ROI THRESHOLD DATA 128 and BRAIN CONDITION DATA 129 combination, either as monitored and adjusted dynamically in a given individual, or through a statistical meta-analysis or other statistical methodology for analyzing the effectiveness of various neuro-cognitive stimulation parameters for a particular ROI THRESHOLD DATA 128 and BRAIN CONDITION DATA 129 across multiple individuals having the same ROI THRESHOLD DATA 128 and BRAIN CONDITION DATA 129 combination. In this manner, the NEURO-COGNITIVE STIMULATION ANALYZER 136 (when embedded and integrated within the post-stimulation feedback loop mentioned above) offers an automatic learning potential for optimizing the neuro-cognitive stimulation parameters for any given ROI THRESHOLD DATA 128 and BRAIN CONDITION DATA 129 combination.

An important aspect of the present invention is the capacity of the BRAIN TRAIT COMPUTATION MODULE 103 to offer a differential diagnostic statistical tool for screening, evaluating, and diagnosing the existence of a particular brain-related disease in an individual at the time of testing, or to offer a reliable predictive diagnostic tool based on statistically reliable deviation of the REGIONS OF INTEREST 124 from the corresponding functional, structural, or cognitive performance distribution in the normal population or sample. In this manner, the BRAIN TRAIT COMPUTATIONAL MODULE 103 may be considered as an independent differential diagnostic tool for assessing the likelihood of an individual being afflicted by a particular brain-related disease, at the time of testing, or prospectively, with a certain probability predictive power, (e.g., in conjunction with the present invention's NEURODIAGNOSTICS MODULE 101, the REGIONS OF INTEREST COMPUTATIONAL MODULE 102, or as constituting an altogether independent differential diagnostic neurobehavioral tool).

More specifically, as the REGIONS OF INTEREST COMPUTATIONAL MODULE 102 may include any one of the three INDIVIDUAL FUNCTIONAL ACTIVATION DATA 116, INDIVIDUAL STRUCTURAL MAPS 118, or INDIVIDUAL COGNITIVE PROFILE in any possible combination or separately—together with the FUNCTIONAL STRUCTURAL COGNITIVE NORM DATA 121, the STANDARD BRAIN REGIONS DEVIATION ANALYSIS 122 is capable of outputting the REGIONS OF INTEREST DATA 124 as either the functional, structural, or cognitive statistically significant deviant features of an individual. Accordingly, the BRAIN TRAIT THRESHOLD COMPUTATION 126 is capable of differentially diagnosing the likelihood of an individual being afflicted with a particular brain-related disease based on functional, structural, or cognitive deviant REGIONS OF INTEREST DATA 124 (separately or together, in any possible combination).

As such, the BRAIN TRAIT COMPUTATION MODULE 103 is also capable of offering a differential diagnostic tool for assessing the likelihood of an individual either being afflicted with a particular brain disease, at the time of testing, or prospectively, within set periods of time based on the INDIVIDUAL FUNCTIONAL ACTIVATION DATA 116, INDIVIDUAL STRUCTURAL MAPS 118, or INDIVIDUAL COGNITIVE PROFILE 120 separately or in any combination. Hence, the BRAIN TRAIT COMPUTATION MODULE 103 may also function as a separate or independent neurobehavioral differential diagnostic tool that is capable of screening the wide population for any existent or prospective brain-related disease (or alternatively for enhanced cognitive performance capabilities in an individual) based on either a simple COGNITIVE DATA 112 (derived from various cognitive or behavioral testing) which is analyzed by the STATISTICAL COMPUTATION MODULE 114 and leads to the INDIVIDUAL COGNITIVE PROFILE 120, or based on more extensive FUNCTIONAL NEUROIMAGING 108 and STRUCTURAL NEUROIMAGING DATA 111 that are analyzed again by the STATISTICAL COMPUTATION MODULE 114 and lead to the INDIVIDUAL FUNCTIONAL ACTIVATION DATA 116 and INDIVIDUAL STRUCTURAL MAPS 118 and the above-mentioned COGNITIVE DATA 112 (in any possible combination).

Indeed, given the low-cost of a preliminary screening testing which obtains only COGNITIVE DATA 112 (which nevertheless can be computed by the STATISTICAL COMPUTATION MODULE 114 and STANDARD BRAIN REGIONS DEVIATION ANALYSIS 122 thereby yielding a statistically significant differential diagnostic or predictive diagnostic capabilities), such cognitive or behavioral testing may be used as an initial wide-population screening tool for the existence or likelihood for the development of various brain-related diseases. Following such low-cost generalized screening testing for the general population for a particular brain disease or diseases (which has a fair-to-good differential diagnostic, or prospective predictive diagnostic power), one could utilize a second-tier, more sophisticated, yet costly, full NEURODIAGNOSTICS MODULE 101 utilization of INDIVIDUAL FUNCTIONAL ACTIVATION DATA 116, INDIVIDUAL STRUCTURAL MAPS 118, and INDIVIDUAL COGNITIVE PROFILE 120 (or any combination thereof) to obtain a much more accurate (with a lower rate of false-positive) differential diagnosis of the particular brain disease.

Another important aspect of the present invention is the capacity of the BRAIN TRAIT COMPUTATION MODULE 103 to offer a predictive statistical tool for screening, evaluating and diagnosing the probability of an individual being gifted in a particular skill or skills or alternatively, diagnosing or assessing the possibility of enhancing a particular cognitive function or functions in an individual, which is computed based on a statistical comparison of the REGIONS OF INTEREST 124 with the corresponding functional structural or cognitive performance distribution in the normal population or sample. The BRAIN TRAIT COMPUTATIONAL MODULE 103 can be considered as an independent differential diagnostic tool for assessing the likelihood of an individual being afflicted with a particular brain related disease or diseases at the time of testing or prospectively with a certain probability predictive power, e.g., in conjunction with the current invention's NEURODIAGNOSTICS MODULE 101, the REGIONS OF INTEREST COMPUTATIONAL MODULE 102, or as constituting an altogether independent differential diagnostic neurobehavioral tool.

More specifically, given the above-mentioned possibility of the REGIONS OF INTEREST COMPUTATIONAL MODULE 102 being any one of the three INDIVIDUAL FUNCTIONAL ACTIVATION DATA 116, INDIVIDUAL STRUCTURAL MAPS 118 or INDIVIDUAL COGNITIVE PROFILE in any possible combination or separately—together with the FUNCTIONAL STRUCTURAL COGNITIVE NORM DATA 121, the STANDARD BRAIN REGIONS DEVIATION ANALYSIS 122 is capable of outputting the REGIONS OF INTEREST DATA 124 as either the functional or structural or cognitive statistically significant deviant features of an individual from cognitively enhanced statistical norms. Accordingly, the BRAIN TRAIT THRESHOLD COMPUTATION 126 is capable of differentially diagnosing the likelihood of an individual possessing either enhanced cognitive function or functions or alternatively sub-enhanced cognitive performance in a particular skill or skills based on functional, structural or cognitive deviant REGIONS OF INTEREST DATA 124 (separately or together in any possible combination). As such, the BRAIN TRAIT COMPUTATION MODULE 103 is also capable of offering a differential diagnostic tool for assessing the likelihood of an individual possessing sub-enhanced (or enhance) cognitive functioning in a particular skill or skills based on the INDIVIDUAL FUNCTIONAL ACTIVATION DATA 116, INDIVIDUAL STRUCTURAL MAPS 118, or INDIVIDUAL COGNITIVE PROFILE 120 separately or in any combination. Hence, the BRAIN TRAIT COMPUTATION MODULE 103 can also function as a separate or independent neurobehavioral predictive assessment statistical tool that is capable of determining whether an individual possesses enhanced cognitive performance capabilities based on either a simple COGNITIVE DATA 112 (derived from various cognitive or behavioral testing) which is analyzed by the STATISTICAL COMPUTATION MODULE 114 and leads to the INDIVIDUAL COGNITIVE PROFILE 120, or based on more extensive FUNCTIONAL NEUROIMAGING 108 and STRUCTURAL NEUROIMAGING DATA 110 that are analyzed again by the STATISTICAL COMPUTATION MODULE 114 and lead to the INDIVIDUAL FUNCTIONAL ACTIVATION DATA 116 and INDIVIDUAL STRUCTURAL MAPS 118 and the abovementioned COGNITIVE DATA 112 (in any possible combination).

Indeed, given the potential low-cost of a preliminary screening testing which obtains only COGNITIVE DATA 112 which nevertheless can be computed by the STATISTICAL COMPUTATION MODULE 114 and STANDARD BRAIN REGIONS DEVIATION ANALYSIS 122 thereby yielding a statistically significant differential predictive assessment capabilities, such cognitive or behavioral testing may be used as an initial wide-population screening tool for the enhanced or sub-enhanced cognitive functioning in any particular skill or skills. It may be the case that following such low-cost generalized screening testing for the general population for a particular cognitively enhanced skill or skills, one could utilize a second-tier more sophisticated yet costly full NEURODIAGNOSTICS MODULE 101 utilization of INDIVIDUAL FUNCTIONAL ACTIVATION DATA 116, INDIVIDUAL STRUCTURAL MAPS 118, and INDIVIDUAL COGNITIVE PROFILE 120 (or any combination thereof) in order to obtain a much more accurate with a lower rate of false-positive differential diagnosis of the particular brain related cognitive enhancement features.

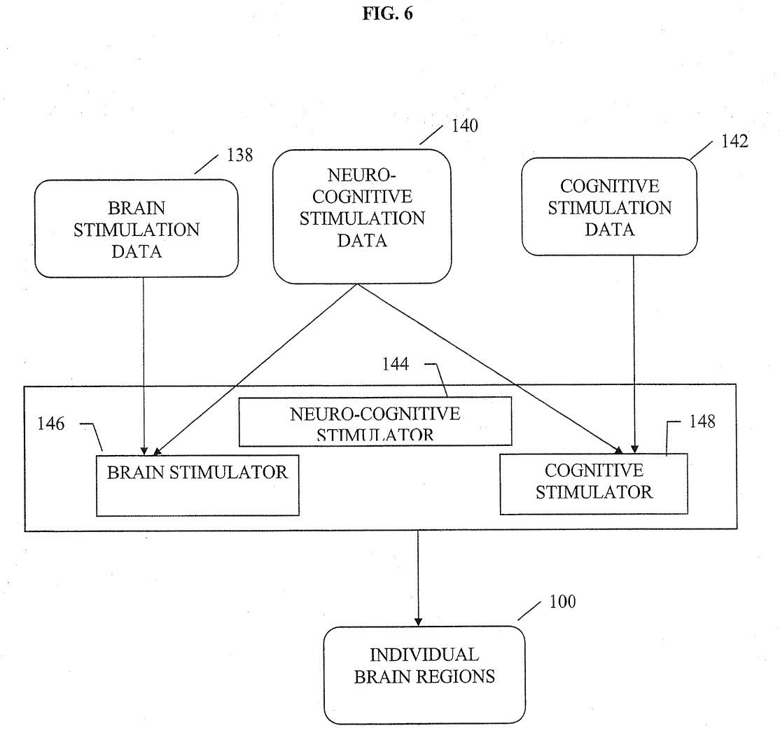

Reference is now made to FIG. 6 which details the STIMULATION MODULE 105 of the system 200 of FIG. 1. The STIMULATION MODULE 105 is configured to stimulate particular brain regions and their corresponding cognitive stimulation in a given individual. The STIMULATION MODULE 105 includes the BRAIN STIMULATION DATA 138, the COGNITIVE STIMULATION DATA 140, and a NEURO-COGNITIVE STIMULATION DATA 140 of FIG. 5, which are input into the NEURO-COGNITIVE STIMULATOR 144. In turn, the NEURO-COGNITIVE STIMULATOR 144 includes a BRAIN STIMULATOR 146 and a COGNITIVE STIMULATOR 148. Specifically, the BRAIN STIMULATION DATA 138 and the NEURO-COGNITIVE STIMULATION DATA 140 are input into the BRAIN STIMULATOR 146, and the NEURO-COGNITIVE STIMULATION DATA 140 and COGNITIVE STIMULATION DATA 142 are input into the COGNITIVE STIMULATOR 148. Based on the BRAIN STIMULATION DATA 138, the COGNITIVE STIMULATION DATA 140, and the NEURO-COGNITIVE STIMULATION DATA 140, the BRAIN STIMULATOR 146 and the COGNITIVE STIMULATOR 148 determine the INDIVIDUAL BRAIN REGIONS 100, which is the actual stimulation of the identified brain region or regions, and which includes an inhibitory or excitatory brain and cognitive stimulation according to particular stimulation parameters determined by the TRAIT-THRESHOLD INVERSE STIMULATION COMPUTATION 132.

An exemplary embodiment of the NEURO-COGNITIVE STIMULATOR 144 includes an integrated BRAIN STIMULATOR 146 and the COGNITIVE STIMULATOR 148, which can stimulate the same INDIVIDUAL BRAIN REGIONS 100 simultaneously or with time-separation between the brain loci and corresponding cognitive stimulation of these brain loci in any possible order. Thus, the NEURO-COGNITIVE STIMULATOR 144 stimulates single or multiple INDIVIDUAL BRAIN REGIONS 100 loci with excitatory or inhibitory brain stimulation parameters including the varying of: the intensity or duration or interval of each of the stimulation brain loci separately or together, while also varying the cognitive “excitatory” or “inhibitory” stimulation of each of these brain loci separately or together (e.g., providing cognitive stimulation or training for each of the stimulated brain regions which corresponds to the excitatory or inhibitory feature of the brain stimulation of a particular loci). For example, an excitatory 10-20 Hz TMS of the left prefrontal cortex aimed at improving or enhancing the mood or well-being of an individual can be coupled with a computerized, auditory, or visual presentation of a Beck-based “positive thinking,” or change in self-construct cognitive stimulation or training paradigm, which may be juxtaposed together in any possible order and with any temporal separation between their onset, termination time, and length of stimulation.

Likewise, an excitatory 10-20 Hz TMS stimulation of the cingulate gyrus geared towards improving concentration or focus, or in conjunction with temporal or hippocampal excitatory 10-20 Hz TMS stimulation to improve deficient memory, executive function, or concentration capabilities or enhance them, can be coupled with a juxtaposition in any temporal order and length or intensity of excitatory cognitive stimulation or training, which may consist of short term memory cognitive exercises or attention allocation exercises. Alternatively, an inhibitory 1 Hz TMS stimulation of diseased Schizophrenic right hemispheric temporal or parietal associated delusional “visions” or “sounds” may be coupled, in any order and temporal length or intensity, with a cognitive stimulation or training geared towards diminishing the likelihood of occurrence of false-perceptions (e.g., through enhanced perceptual training such as enhancing perceptual cues in perceptual illusion paradigms or other perceptual paradigms or, alternatively, through enhancing accurate perception training or through cognitive stimulation or training in enhancing attention or attentional allocation capabilities, or increasing psychophysical judgment capabilities).

Alternatively, individuals who have been characterized as possessing functional, structural, or cognitive abnormalities that are characteristic of autism may be stimulated by the NEURO-COGNITIVE STIMULATOR 144 through a combination of excitatory 10-20 Hz TMS stimulation of the LH's Broca's and Wernicke's regions and an inhibitory 1 Hz TMS of the abnormally hyperactivated (or structurally enlarged) contralateral RH's Broca's and Wernickes' language regions, that are coupled with cognitive or behavioral stimulation geared towards enhancing language development, articulation, naming, pointing, or joint attention skills, among others.

In yet another exemplary embodiment, the NEURO-COGNITIVE STIMULATOR 144 can also facilitate neuroplasticity changes geared towards improving functional, structural, or corresponding cognitive performance capabilities associated with a particular brain disease or, alternatively, geared towards enhancing a particular cognitive function or functions through an excitatory or inhibitory brain stimulation of single or multiple INDIVIDUAL BRAIN REGIONS 100 brain loci, which is combined with “opposite direction” inhibitory or excitatory cognitive stimulation. In yet another embodiment, the NEURO-COGNITIVE STIMULATOR 144 may enhance a particular cognitive function or functions through an excitatory or inhibitory brain stimulation of single or multiple INDIVIDUAL BRAIN REGIONS 100 brain loci which is combined with apparently “opposite direction” inhibitory or excitatory cognitive stimulation.

An example of such “opposite-direction” brain stimulation and cognitive stimulation can be the inhibitory 1 Hz TMS brain stimulation of the Amygdala or fusiform gyrus (which have been shown to be hyperactivated in ASD individuals during facial recognition and social cognition tasks, or during non-social communication paradigms or even at resting conditions) during resting conditions or during the conductance of non-social cognition tasks—which may be coupled with focused social cognition stimulation exercises (before or after the inhibitive TMS stimulation during the resting state or non-social communication tasks). Alternatively, the NEURO-COGNITIVE STIMULATOR 144 may activate the BRAIN STIMULATOR 146 or COGNITIVE STIMULATOR 148 separately, or with opposite excitatory vs. inhibitory stimulation parameters, for the same or different brain loci at the same or different time points or intervals.

The NEURO-COGNITIVE STIMULATOR 144 is also capable of dynamically adjusting or altering the intensity or interval of brain or cognitive stimulation of single or multiple INDIVIDUAL BRAIN REGIONS 100 brain loci, or the temporal juxtaposition of single or multiple brain stimulation loci and their corresponding cognitive stimulation based on potential changes in the TRAIT-THRESHOLD INVERSE STIMULATION COMPUTATION 132 that can arise as a result of the post-stimulation feedback measurement by the NEURODIAGNOSTICS MODULE 101 and subsequent computations by the REGIONS OF INTEREST COMPUTATIONAL MODULE 102, the BRAIN TRAIT COMPUTATION MODULE 103, and the TREATMENT MODULE 105.

In yet another embodiment, the NEURO-COGNITIVE STIMULATOR 144, the BRAIN STIMULATOR 146 and the COGNITIVE STIMULATOR 144 form a single integrated medical device, which is capable of synchronizing the brain stimulation of single or multiple brain INDIVIDUAL BRAIN REGIONS 100 loci together with the cognitive stimulation of the same brain loci, which may be controlled by the TRAIT-THRESHOLD INVERSE STIMULATION COMPUTATION 132 output BRAIN STIMULATION DATA 138, the COGNITIVE STIMULATION DATA 140, and the NEURO-COGNITIVE STIMULATION DATA 140. Alternatively, the NEURO-COGNITIVE STIMULATOR 144 can include at least two separate medical devices of the BRAIN STIMULATOR 146 and the COGNITIVE STIMULATOR 148 that are controlled by the same TRAIT-THRESHOLD INVERSE STIMULATION COMPUTATION 132 through its output of the BRAIN STIMULATION DATA 138, the COGNITIVE STIMULATION DATA 140, and the NEURO-COGNITIVE STIMULATION DATA 140.

The COGNITIVE STIMULATOR 148 may be of single or multiple presentation of various sensory modality stimulation such as visual, auditory, and tactile, for example, with various response modalities being used in any possible combination, including but not limited to a keypress response, vocal, written, tactile, or visually guided response with or without a response feedback element (e.g., which provides a feedback as to the accuracy of the subject's response or performance at different time points, or with regards to various segments of the task or tasks at hand).

The BRAIN STIMULATOR 146 may include a medical device capable of stimulating electromagnetically, electrically, magnetically, and/or photoelectrically, and inhibitorily or excitatorily, a single or multiple INDIVIDUAL BRAIN REGIONS 100 brain pixels, regions, tissues, functional neural units, or hemispheres, which have been deemed as functionally, structurally, or cognitively diseased by the BRAIN TRAIT THRESHOLD COMPUTATION 126 and based on the control of the BRAIN STIMULATION ANALYZER 133 and the direct input of the BRAIN STIMULATION DATA 138. Alternatively, the BRAIN STIMULATOR 146 may be a medical device capable of stimulating electromagnetically, electrically, magnetically, or photoelectrically, a single or multiple brain pixels, regions, tissues, functional neural units or hemispheres, which are functionally or structurally associated with a particular sub-enhanced cognitive function or functions by the BRAIN TRAIT THRESHOLD COMPUTATION 126 and based on the control of the BRAIN STIMULATION ANALYZER 133 and the direct input of the BRAIN STIMULATION DATA 138.

In yet another embodiment, the BRAIN STIMULATOR 146 may include a medical device capable of stimulating electromagnetically, electrically, magnetically, and/or photoelectrically, and inhibitorily or excitatorily, a single or multiple brain pixels, regions, tissues, functional neural units, or hemispheres through the convergence of at least two electrical, magnetic, electromagnetic, or photoelectric sources of energy or stimulation, in any possible combination. These single or multiple electrical, magnetic, electromagnetic, or photoelectric sources can be placed at any point on top of the cranium or surface of the scalp, or face or neck, broadly defined or non-invasively within any of the orifices located in the head, e.g., the ears, nose, sinuses, mouth and larynx, eyes. Additionally, each of these stimulating or receiving electrical, magnetic, electromagnetic, or photoelectric sources is controlled individually or collectively by the NEURO-COGNITIVE STIMULATOR 144 and specifically through the dynamic input from the BRAIN STIMULATION DATA 138.

Following the ROI NEUROCOGNITIVE STIMULATION 150, feedback measurements are performed by the NEURODIAGNOSTICS MODULE 101, REGIONS OF INTEREST COMPUTATIONAL MODULE 102, BRAIN TRAIT COMPUTATION MODULE 103, TREATMENT MODULE 104, and STIMULATION MODULE 105, as depicted in FIG. 1, and as detailed above. The inclusion of such a “feedback loop” (i.e., from the STIMULATION MODULE 105 to the NEURODIAGNOSTICS MODULE 101) allows to monitor and adjust the individual disease-based or cognitive enhancement stimulation parameters continuously following stimulation. It also allows for a dynamic automatic learning taking place at the TREATMENT MODULE 104, i.e., in terms of the TRAIT-THRESHOLD INVERSE STIMULATION COMPUTATION 132 optimization—for a particular disease, or individual based on a comparison of the pre- and post-stimulation ROI THRESHOLD DATA 128 and BRAIN CONDITION DATA 129 (namely, a statistical meta-analysis or any other statistical procedure which is capable of cumulatively assessing the relationship between varying the pre-stimulation parameters output by the BRAIN STIMULATION ANALYZER 133, COGNITIVE STIMULATION ANALYZER 134, or NEURO-COGNITIVE STIMULATION ANALYZER 136 for a specific BRAIN CONDITION DATA 129 disease or particular cognitive enhancement protocol and particular ROI THRESHOLD DATA 128 and the post-stimulation measured ROI THRESHOLD DATA 128 and BRAIN CONDITION DATA 129, to determine the most effective brain stimulation, cognitive stimulation and corresponding neuro-cognitive stimulation parameters).

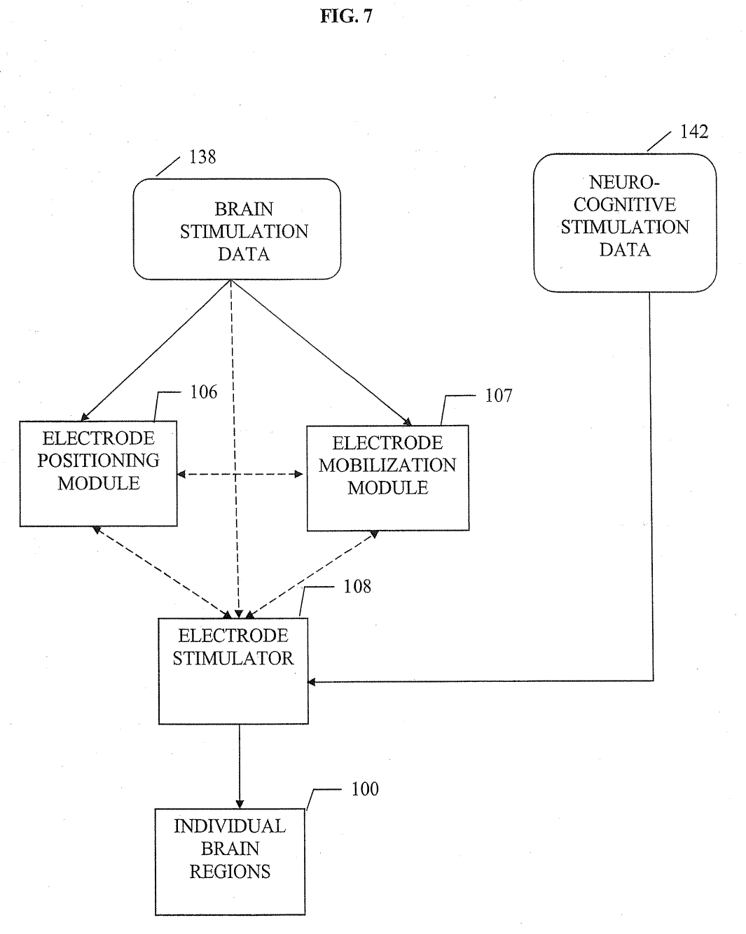

Reference is now made to FIG. 7 which details the BRAIN STIMULATOR 146 of FIG. 6. The BRAIN STIMULATOR 146 is configured to stimulate particular single or multiple brain loci based on input from the BRAIN STIMULATION DATA 138 and NEURO-COGNITIVE STIMULATION DATA 140, which outputs to the ELECTRODE MOBILIZATION MODULE 107 information regarding the positioning, loci, axis of stimulation, and direction of the ELECTRODE STIMULATOR 108 for stimulation of single or multiple brain loci. The ELECTRODE MOBILIZATION MODULE 107 receives, in turn, monitoring of the current localization, axis, stimulation direction and brain regions, which are input into the ELECTRODE STIMULATOR 108. The ELECTRODE POSITIONING MODULE 106 continuously assists the ELECTRODE MOBILIZATION MODULE 107 to bring the electrodes (or any other electrical or electromagnetic stimulation device) to a position and axis of stimulation or precise localization of stimulation to the determined single or multiple brain regions INDIVIDUAL BRAIN REGIONS 100. Once the ELECTRODE STIMULATOR 108 is positioned in such single or multiple brain localizations, which allows for the stimulation of the desired single or multiple INDIVIDUAL BRAIN REGIONS 100 as determined through the continuous interaction between the ELECTRODE MOBILIZATION MODULE 107 and the ELECTRODE POSITIONING MODULE 106, then the ELECTRODE STIMULATOR 108 stimulates the desired INDIVIDUAL BRAIN REGIONS 100.

The physical engineering or configuration of the ELECTRODE STIMULATOR 108 may be such that it requires little or no physical mobilization by the ELECTRODE MOBILIZATION MODULE 107, but instead is activated based on the BRAIN STIMULATION DATA 138. An example of such an embodiment includes an ELECTRODE STIMULATOR 108 which comprises numerous multiple electromagnetic, magnetic, electrical, and/or photoelectrical stimulators placed at multiple locations on top of the scalp or within the mouth, nose, eyes, or ear cavities and each controlled by a computer signal which allows for the rotation of their electromagnetic or electrical direction, or axis of stimulation or region or regions which are stimulated by each of them. Additionally, the ELECTRODE STIMULATOR 108 may be constructed such that it sends and receives electrical, electromagnetic, magnetic and/or photoelectrical signals (or any combination of them) between electrodes. The ELECTRODE STIMULATOR 108 may also comprise magnetic, electric, electromagnetic and/or photoelectric stimulators placed at any of the locations mentioned above, and controlled by a mutual computer, which therefore allows for the convergent or emission or receptive stimulation of any single or multiple points, locus or loci, region or regions, of the brain.

The functioning of the BRAIN STIMULATOR 146 in terms of its ongoing and continuous stimulation of the desired INDIVIDUAL BRAIN REGIONS 100 may be continuously adjusted to simulate the same or different INDIVIDUAL BRAIN REGIONS 100 based on the above-mentioned described invention and depicted in FIG. 1. As such, the BRAIN STIMULATOR 146 can serve as a means for treating various brain-related diseases such as Alzheimer's, depression, autism, and other diseases mentioned above, or can serve as a means for enhancing particular cognitive functions or skills in a normal individual.

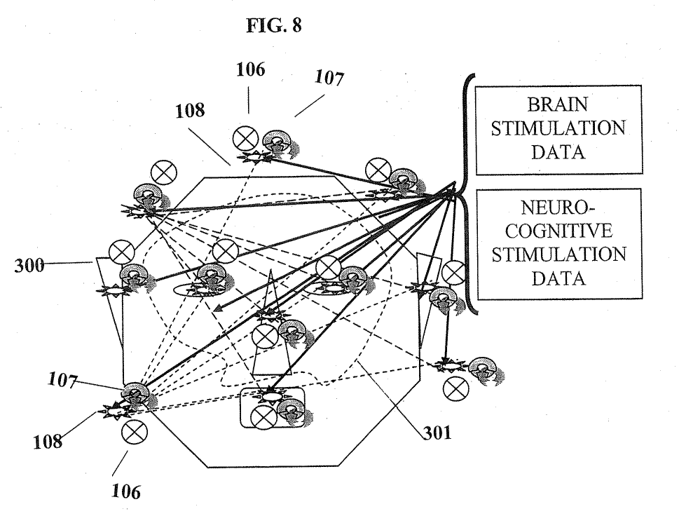

Reference is now made to FIG. 8 which details another schematic representation of the BRAIN STIMULATOR 146 of FIG. 6. The BRAIN STIMULATOR 146 is in the form of a helmet or similar device 300 (shown schematically in FIG. 8 as covering at least part of an individual's head 301) including single or multiple ELECTRODE STIMULATOR 108 which are electrical or electromagnetic stimulating agents capable of stimulating single or multiple brain regions, points, cells, lobes, or hemispheres. The ELECTRODE STIMULATORs 108 are controlled by both the BRAIN STIMULATION DATA 138 and the NEURO-COGNITIVE STIMULATION DATA 142 of FIG. 7. Each of the single or multiple ELECTRODE STIMULATOR 108 is also being evaluated by an adjacent or associated ELECTRODE POSITIONING MODULE 106, which can determine the location of each of these ELECTRODE STIMULATORs 108 relative to a person's individual brain structure, and their respective regions which can be stimulated by the ELECTRODE STIMULATOR 108 in this position or axis of stimulation. This individual brain specific localization of each ELECTRODE STIMULATORs 108 is then utilized along with stimulating-agent specific input from the BRAIN STIMULATION DATA 138, and the NEURO-COGNITIVE STIMULATION DATA 142 is output to the ELECTRODE POSITIONING MODULE 106 to adjust the localization, axis of stimulation or specification of the direction, or regions, cells, lobes, or hemispheres or any specification of a single or multiple brain points or locations by the ELECTRODE MOBILIZATION MODULE 107. The ELECTRODE MOBILIZATION MODULE 107 sends, in turn, feedback to the ELECTRODE POSITIONING MODULE 106, thereby allowing for a continuous adjustment and optimization of the precise localization of each of the ELECTRODE STIMULATOR 108 so that it is capable of stimulating all of the determined single or multiple INDIVIDUAL BRAIN REGIONS 100. Once each of these ELECTRODE STIMULATORs 108 has been determined by its accompanying ELECTRODE POSITIONING MODULE 106 to be located in the appropriate position so as to stimulate the corresponding single or multiple INDIVIDUAL BRAIN REGIONS 100 based on the corresponding BRAIN STIMULATION DATA 138, and based on the input from the NEURO-COGNITIVE STIMULATION DATA 142, the single or multiple ELECTRODE STIMULATOR 108 begin stimulating the determined INDIVIDUAL BRAIN REGION 100 in conjunction with the COGNITIVE STIMULATOR 148 of FIG. 6.

As depicted in FIG. 8, the ELECTRODE STIMULATORs 108 of device 300 may be placed over the scalp, head, face, neck, or within the eyes, ears, mouth, or nose cavities or orifices/spaces, and which may be either rotated or mobilized or otherwise change their stimulation direction of different single or multiple brain localizations or regions or points. Their convergence or emission and reception by different such ELECTRODE STIMULATORs 108 allow for the stimulation of any three-dimensional point or points, cells, tissue, region, lobe, or hemisphere within an individual's brain and can be individually controlled for each of these ELECTRODE STIMULATORs 108 based on the input from the BRAIN STIMULATION DATA 138 and in conjunction with the NEURO-COGNITIVE STIMULATION DATA 142, to treat any brain-related disease or enhance any cognitive function or functions in an individual.

An exemplary embodiment of the BRAIN STIMULATOR 146 includes multiple ELECTRODE STIMULATORs 108 that are placed individually over the teeth of a person, or placed anywhere else within the mouth cavity, throat, ears, nose, eyes and on the surface of the scalp, face, neck in a manner which allows for the ELECTRODE MOBILIZATION MODULE 107 to change, alter, or control the direction of electrical or electromagnetic or any combination of these two types of stimulations of each of these specific ELECTRODE STIMULATORs 108, in a manner that allows for each ELECTRODE STIMULATORs 108 to send, transmit, or receive such stimulation through any single or multiple brain point or points or regions, and wherein the precision of any line or slice or direction or region of stimulation may be made more precise or accurate due to the convergence of stimulation from multiple ELECTRODE STIMULATORs 108 or through an emission and reception of electrical or electromagnetic stimulation by single or multiple such ELECTRODE STIMULATORs 108. As a result, the whole brain becomes a field of numerous multiple points, lines, spheres, regions, organs, lobes, cells, or hemispheres of potential stimulation by convergence or by emission and reception of single or multiple such ELECTRODE STIMULATORs 108, which are controlled by the input from the BRAIN STIMULATION DATA 138 and based on the above-mentioned invention.

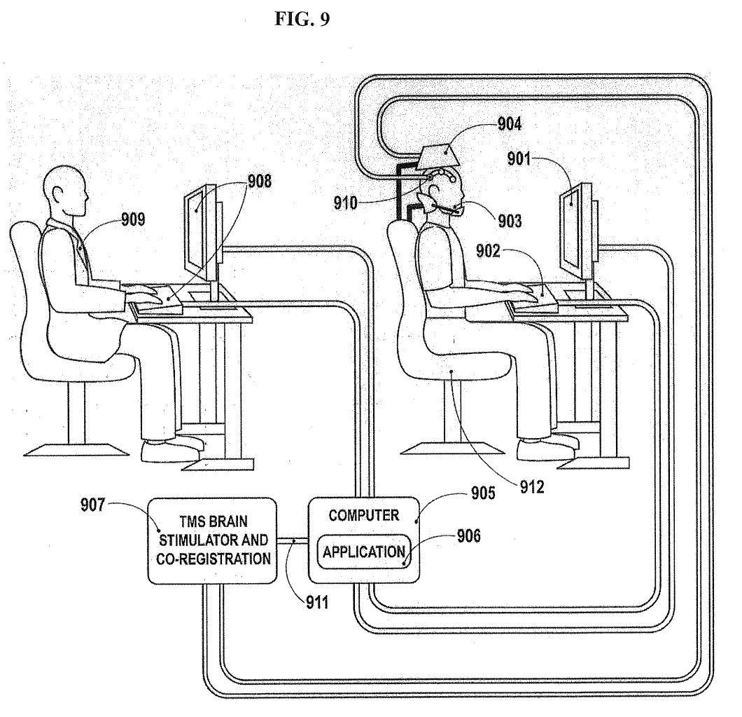

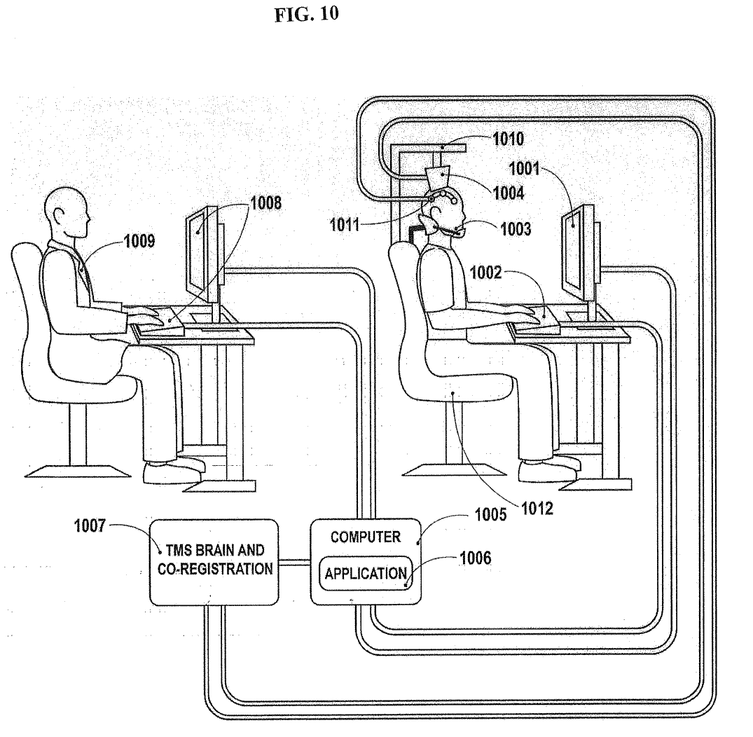

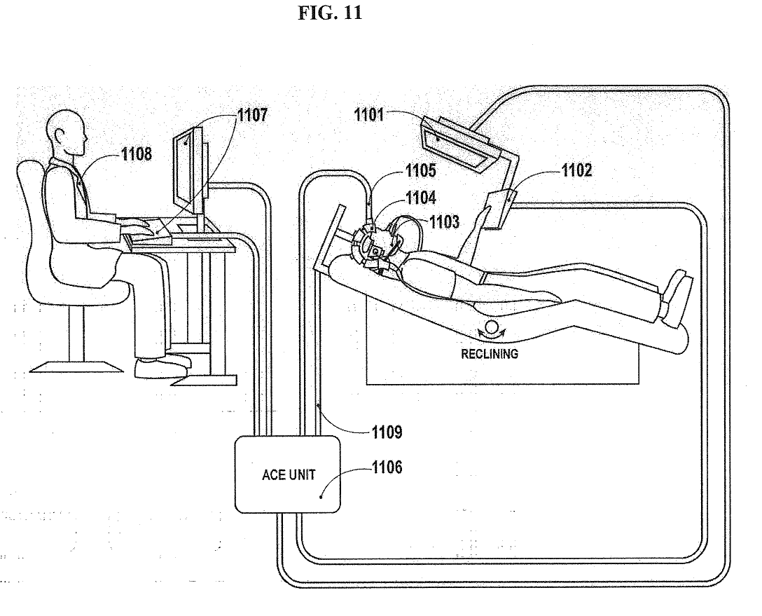

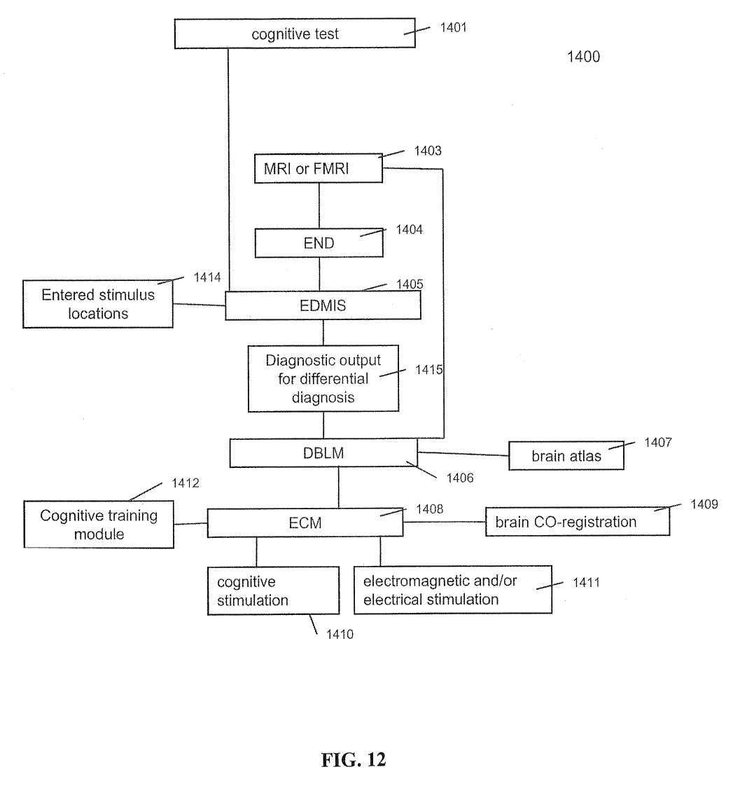

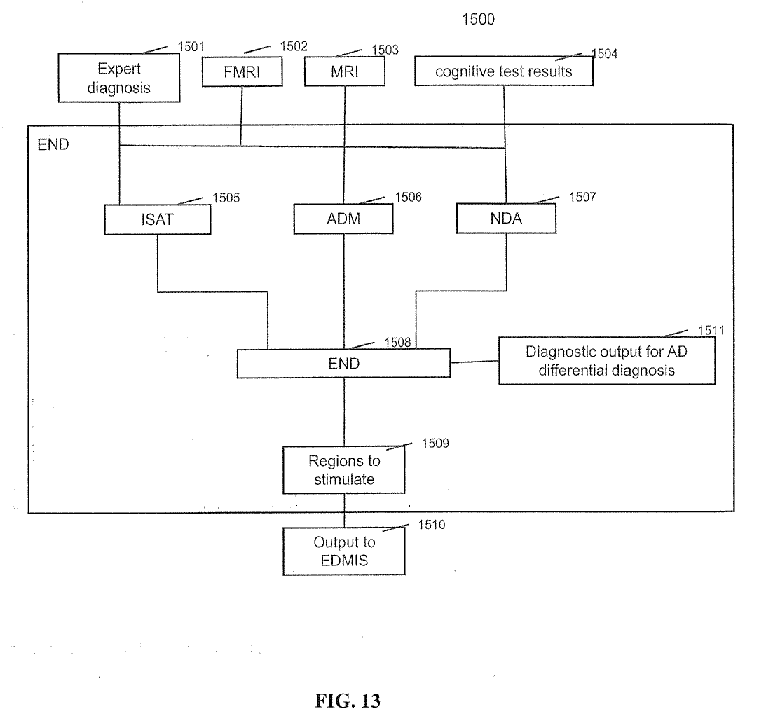

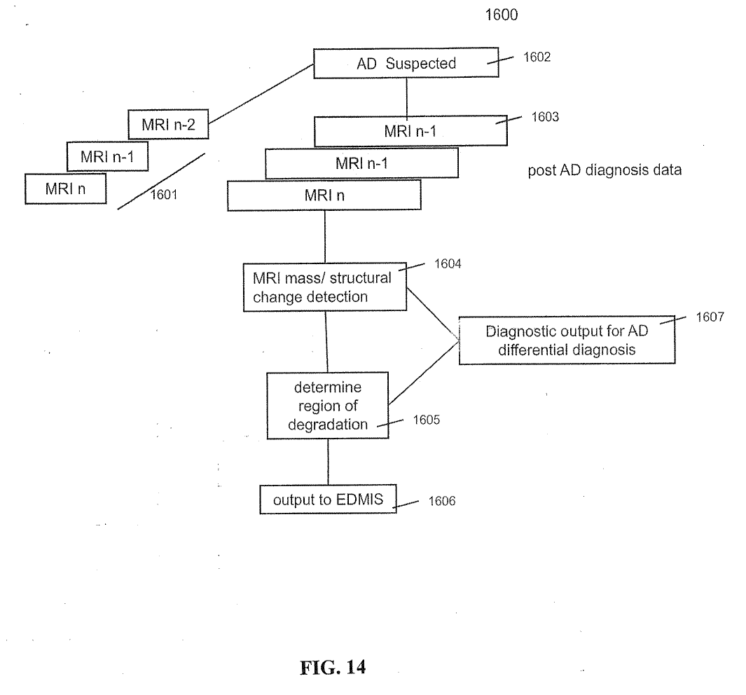

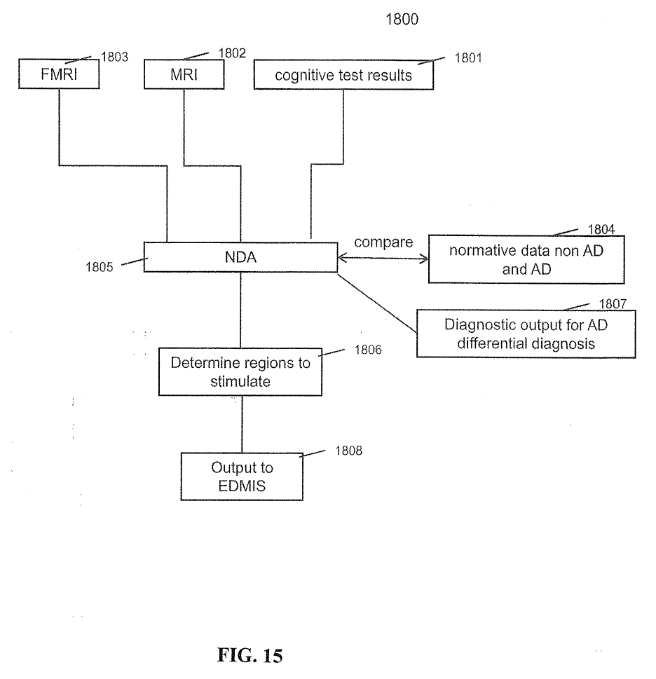

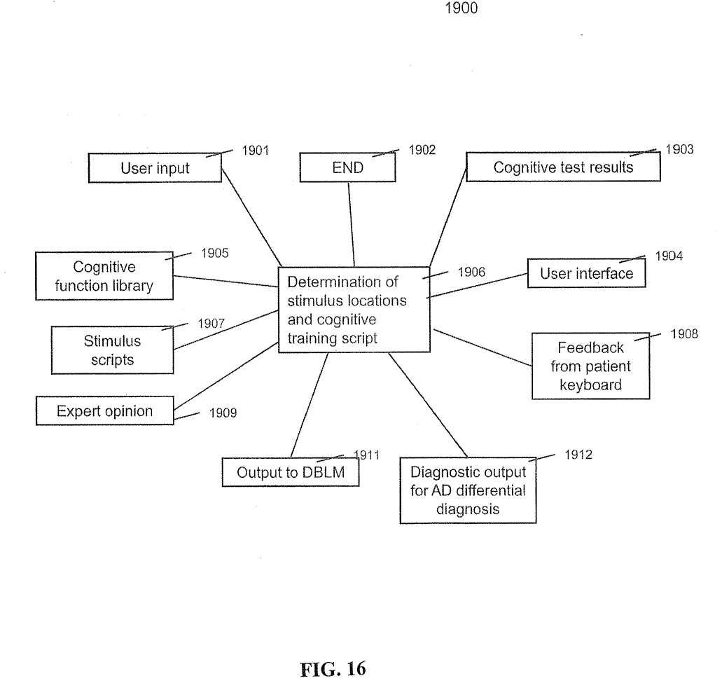

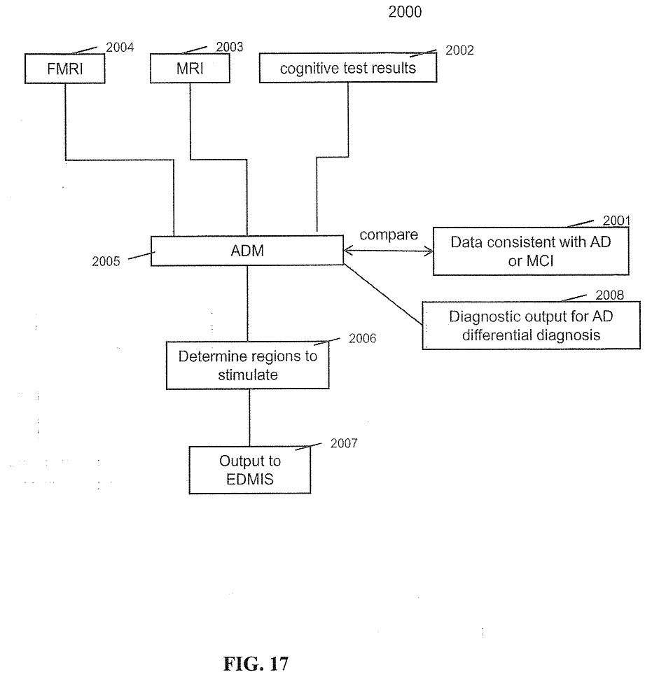

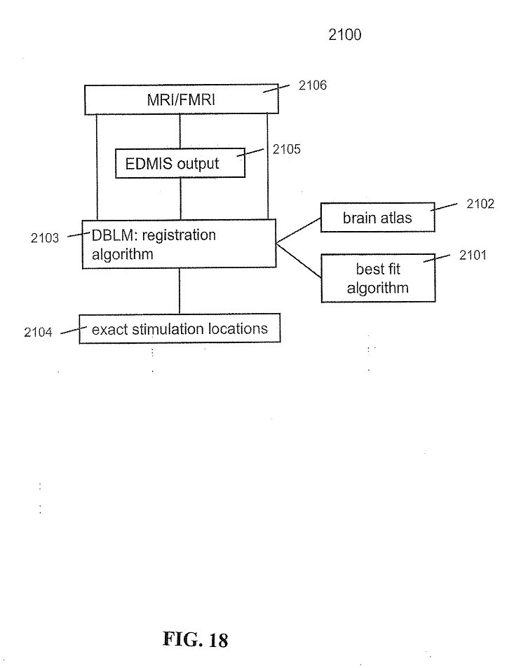

The invention will now be described with reference to FIGS. 9-20 and Embodiments A-C, variations and enhancements thereof.

### EMBODIMENT A

The system of Embodiment A includes the elements as described in the following description and with references to FIG. 9-13. Embodiment A can provide a synchronized TMS magnetic stimulus and Cognitive training stimulus to the patient at locations identified by the care provider or algorithmically identified Alzheimer's diseased brain regions. The system may include a computer, a TMS Stimulator (905, 907), a housing unit suitable for a patient, with a TMS coil (904).

The computer can include two screens (901, 908) and keyboards (902, 908), one (908) that allows interaction with the operator (909), and the other (901) supplying cognitive stimuli and commanded by the Executive Control Module (ECM). The patient (910) can provide feedback to the computer (905) using a keyboard (902). In addition, the computer (905) instructs the TMS unit (907) to output a pulse utilizing a connection between the two units (911) within a pre-defined time period after the application of TMS stimulation.

The patient (910) is seated in a comfortable chair (912). The chair allows for seating in an upright or reclining position. The patient's head may be restrained from motion using a restraint (903) and the housing unit (904) may be secured to the patient using appropriate fastening techniques.

The TMS magnetic stimulus is applied to the patient (910) using the housing unit via TMS coil (904). The TMS coil is temperature controlled. The TMS magnetic stimulus units are discussed in more detail below.

The computer application (906, 1400) for the system of the exemplary Embodiment A of the present invention can provide the following functions, the details of preferred component modules being separately described herein below. The Executive Control Module is responsible for managing the sequencing and state of the treatment session (1408) and application of stimuli. The ISAT (1505) component of END (1404, 1505) uses sequences of MRI images to identify changes in brain mass or structure over time. The ISAT (1505) component of END (1404, 1505) may also utilize any of the other END alternatives (NDA or ADM) in any combination. The EDMIS module (1405) uses cognitive test results, the output from END (1404) and input from the caregiver or offsite personnel, to determine the best stimulation locations and training regime, based on stored scripts (1413). The Cognitive Training Module CSM (1412) applies stimulus to the patient (910) based on dynamically alterable scripts (1413). The Diseased Brain Localization Module (DBLM) (1406) takes the location identified by EDMIS (1405) and correlates the identified location for a specific patient's anatomy and locates the correct stimulation locations based on a brain atlas (1407). The Brain Co-Registration Component (907, 1409) determines the exact coordinates of the location to be stimulated on the patient and indicates and controls the registration between the TMS coil location (904), the applied magnetic pulse and the patient's desired stimulus location.

According to another exemplary embodiment of the present invention, an option to Embodiment A may be tailored towards enhancing cognitive functions in normal individuals, for example, by essentially replacing the EDMIS with an equivalent module which is termed Enhanced Cognitive Functions Decision Making System (ECFDM). This module would similarly identify the specific brain region/s which should be stimulated in order to enhance a particular cognitive function or functions or skill/s. based on the input of the END and Cognitive Testing Module, and which is similarly connected to the Executive Control Module which then coordinates (and synchronizes) between the delivery of electromagnetic and cognitive stimulation to the ECFDM's identified brain region/s or loci which need to be stimulated in order to enhance the particular cognitive function/s in a normal individual.

During the treatment or after any single or multiple sessions using stimulation from the system of Embodiment A, the EDMIS (1405)—based on patient's response (902) or based on changes in the patient's brain structure, function, neuroplasticity, or neurophysiology etc. as continuously or intermittently measured by the END—ISAT (1505), NDA (1507) or ADM (1506)—makes determinations based on that response, alerting the operator (909) or modifying the script (1413) as required to optimize cognitive training.

In this embodiment, a feedback loop measures the patient's functional or structural or neuroplasticity or neurophsyiological state (e.g., in terms of degenerative or post-stimulation regenerative/neuroplasticity changes across time, ISAT; or relative to the normal age, education, or other parameters matching population, NDA; or relative to Alzheimer's diseased or relative to any other brain diseased population) prior to single or multiple sessions of electromagnetic and/or cognitive stimulation and also following such single or multiple treatment sessions. This feedback loop utilizes repeated measurements by the END (ISAT, NDA or ADM) and accordingly the EDMIS adjusts the parameters of brain stimulation locus/loci, intensity, duration, frequency etc. and may also adjust the corresponding Cognitive Stimulation of these electromagnetically stimulated brain regions.

### EMBODIMENT B

An enhancement to the functionality of the system of Embodiment A is the system of Embodiment B which adds the following functions: the full functionality of the END module, the preferred embodiment of which is described in detail below. The END module utilizes one or more of the following algorithms for determination of stimulus locations:

Inter Subject Across Time (END-ISAT) (1505, 1600).

Normative Data Analysis (END-NDA) (1507, 1800).

Alzheimer's Diagnostic Module (END-ADM) (1506, 2000).

The system of Embodiment B may further add computer control of the magnetic stimulation (1010). This feature may be implemented in a closed-loop method by utilizing the functionality of the Brain Co-Registration.

### EMBODIMENT C

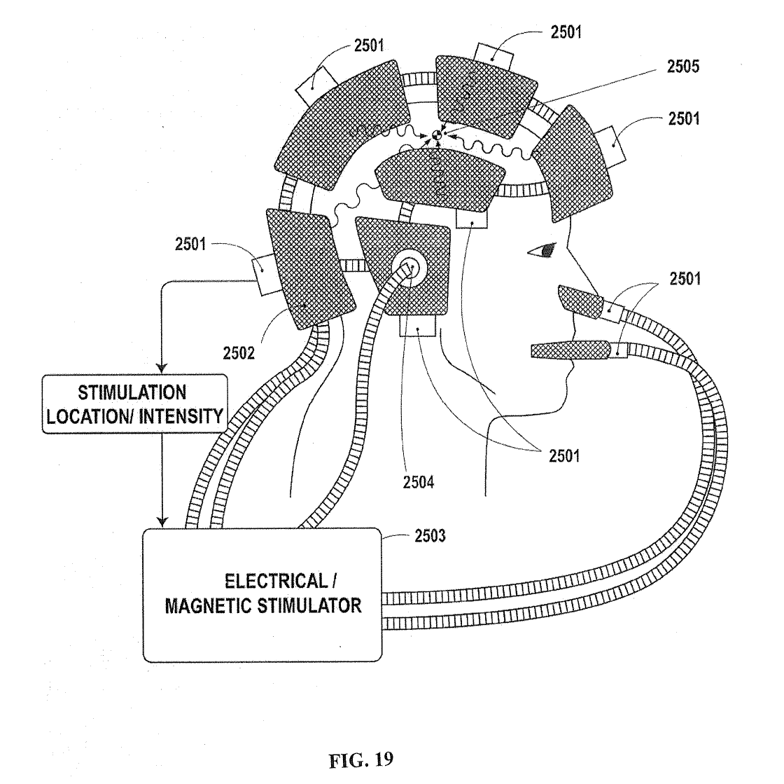

An enhancement to the functionality of the system of Embodiment B is the system of Embodiment C, which adds the following components and functions, including the stimulator illustrated in FIG. 19. The stimulator of FIG. 19 provides enhanced stimulation of the brain regions by utilizing electrical, electromagnetic, magnetic, or a combination of any or all of these. This stimulation may include multiple coils, surface electrodes, and implanted neuronal electrodes, or a combination of any or all of these, placed around the patient's head and in the cavities of the patient's head invasively or non-invasively (2501), to optimize the intensity of targeting a particular brain region (2505).

The stimulator of FIG. 19 includes a helmet and or frame (2506) with coil position control and stabilization utilizing positional feedback as well as rate feedback mechanisms such as gyroscopic position sensors and gyroscopic stabilization systems (2501), in order to optimize and control stimulation location precisely and automatically. The gyroscopic components can continuously sense, adjust, mobilize and control the location and vector of each of the magnets or electrodes of the helmet or frame (2506).

The stimulator of FIG. 19 provides vector magnitude and direction control of the applied magnetic field relative to the patient's head or brain regions by providing feedback to the stimulation controller (2503), and can include cooling and thermal management (1105). In addition, the stimulator of FIG. 19 includes adjustment of Stimulation Location and Intensity with tracking to Norm or Other indicator as Feedback.

The stimulator of FIG. 19 is capable of manual or computer control of the stimulation coils and electrodes (2502, 1104), positioning actuators, and sensors. Under the Computer Control (1106), the system provides real time feedback for stimulation location and intensity, and provides for correction as required.

The Computer Application of the system of Embodiment C is enhanced by the following features:

a. Magnetic Field control module for controlling the applied magnetic field vector using the stimulator of FIG. 19.

b. Electrical Stimulation Pulse Module for controlling and applying electric stimulus both invasively and non-invasively.

c. Gyroscopic Control Module which monitors the feedback of the Inertial sensors and controls the gyroscopic stabilization of the stimulator of FIG. 19.

d. Cognitive Progress Monitoring during the treatment session in the form of tracking of cognitive test results during the treatment session by the computer application of Embodiment C, allowing real time assessment of cognitive function during the treatment session using feedback to the EDMIS module.

e. Database storage and retrieval of data gathered during the session, including patient stimulation location accuracy, patient stimulation levels and cognitive training results.

According to an exemplary embodiment of the present invention, the system of Embodiment C described above includes all of the subsystems as described in the embodiments below.

System Subcomponents

The Executive Control Module (ECM):

The ECM (1408) may be a component of a computer application (1400) that controls the application of excitation stimulus (1411) and cognitive stimulus (1410). The ECM can: (1) manage gathering stimulation location input data from the DBLM (1406); (2) sequence the application of the TMS applied stimulation and the cognitive stimulation to the patient at location(s) specified by the DBLM (1406); and/or (3) monitor the output of the EDMIS (1405) and DBLM (1406), in order to provide modification to the treatment profile, as determined by EDMIS and DBLM.

The ECM (1408) can: (4) time the cognitive stimulus (1410) for about 50 to 500 mSec after the excitation stimulus (1411); (5) provide a trigger output to the TMS unit in order to command application of the applied TMS pulse (1410); and (6) utilize the Brain Co-Registration (1409) module to identify the ideal location of coil (904, 1004 and 1104) and control and locate TMS stimulus (1410).

Further, the ECM (1408) can indicate incorrect placement of the coils, or use computer controlled positioning (1010) to correct the stimulus location and communicate with the CSM (1412) in order to coordinate and control cognitive stimulation to the patient.

The END Module (1500):

The END Module (1500) includes a set of algorithms to determine the presence of Alzheimer's disease (AD). These algorithms may be part of a larger application, or a separate diagnostic application which in combination with EDMIS (1405) can be utilized for early or late stage diagnosis of disease. The END Module accepts input in the form of MRI (1503) or FMRI (1502) data, expert diagnosis (1501) or Cognitive Test Results (1504), and outputs diagnostic output for AD differential diagnosis (1511, 1607, 1807, 2008). The END module uses one or more the following algorithms for determination of stimulus locations:

Inter Subject Across Time (END-ISAT) (1505, 1600):

The ISAT may be implemented as a computer algorithm in an application (1400) and uses Multiple MRI images (1601) acquired over a time period, taking at time intervals to determine brain tissue mass or structural changes indicative of Alzheimer's disease. The ISAT module (1505, 1600) takes the MRI (1601) and performs rotation and scaling to achieve the best correlation between the images. The ISAT module (1505, 1600) also differences the images, as well as differences high pass filtered or edged enhanced images in order to locate structural changes and mass changes in the brain. The ISAT module (1505, 1600) indicates the location of suspected areas of change to the user, allowing the user to input, review, and enter or modify the treatment locations. The ISAT module also reads MRI data from industry standard MRI equipment (1503).

The ISAT output (1606) indicates specific brain regions to be stimulated and includes a tracking index for each region, allowing quick determination of degradation or improvement.

Normative Data Analysis (END-NDA) (1800):

The NDA (1805) is implemented as a computer algorithm and utilizes MRI (1802) and FMRI (1803) data, or cognitive test results (1801). NDA (1805) compares the following indicators of disease to normative values (1804), derived from analysis of industry accepted norms, or norms developed by the applicant. The NDA normative data (1804) is age-matched to the patient. The NDA (1805) scales, rotates and normalizes the data, for comparison to an internally sorted representation of normal subject structure and mass of the same age (1806).

The NDA (1804) uses an algorithm consisting of differencing of data between the applied scaled, rotated and intensity-normalized image, and the reference image, comparing the differenced data to a predetermined threshold, that threshold being determined by comparison of normalized normal patient data, to patient data from diseased brain tissue.

The NDA disease determining threshold is a spatial threshold in 3 degrees of space, consisting of a 4-dimensional value. The NDA contains multiple thresholds, based on the type of disease, or the level of disease progress to be identified. These NDA utilize multiple thresholds to calculate a disease progression gradient, marking on the output, the magnitude and direction of disease progression, indicating that calculated index and identified area of the brain to the EDMIS algorithm (1808). The NDA output data may be used on its own, to identify and track disease progress for diagnostic purposes. The NDA module may optionally accept input form cognitive performance measures.

The Alzheimer's Diagnostic Module (END-ADM) (2000):

The ADM (2005) may be implemented as a computer algorithm. The ADM (2005) indicates the presence of disease at very early stages, ideally about 4 to about 10 years prior to onset. The output of the ADM is the diseased brain regions to be stimulated (2006). The ADM utilizes MRI (2003), FMRI (2004) and cognitive test results data (2002) gathered during FMRI (2004) imaging. The ADM (2005) determines diseased brain regions by analysis against properties associated with Alzheimer's disease or MCI patients (2001). The ADM (2005) scales, rotates and normalizes the data, for comparison to an internally sorted representation of diseased subject structure and mass (2008).

The ADM (2005) uses an algorithm consisting of differencing of data between the applied scaled, rotated and intensity normalized image, and the reference image, comparing the differenced data to a predetermined threshold, that threshold being determined by comparison of normalized diseased patient data.

The ADM disease determining threshold is a spatial threshold in 3 degrees of space, consisting of a 4-dimensional value. The ADM (2005) contains multiple thresholds, based on the type of disease, or the level of disease progress to be identified. These ADM (2005) utilize multiple thresholds to calculate a disease progression gradient, marking on the output, the magnitude and direction of disease progression, indicating that calculated index and identified area of the brain to the EDMIS algorithm (2007). The ADM (2005) output data may be used on its own, to identify and track disease progress for diagnostic purposes (2008). The ADM norm thresholds is calculated from the ADNI database, external databases, or other AD indicative data (2001).

The output of the ADM (2005) is the diseased brain regions (2006) which can be utilized either for diagnosing the disease up to about 4 to about 10 years prior to clinical symptoms, or for therapeutically stimulating these diseased brain regions.

The Diseased Brain Localization Module (DBLM) (2100):

The DBLM (2100) may be implemented as a software module or computer application. The DBLM (2100) identifies the diseased location of the brain based on the brain atlas (2102) and the patient's MRI (2106). The DBLM (2100) allows the user to indicate the location of the brain to be stimulated (2104), by allowing the user to click a computer “mouse” on an image of a representative brain, or on a reconstructed MRI image from the patient. The DBLM (2100) receives input from the EDMIS (2105), to establish treatment locations for a specific patient. The DBLM (2100) interfaces to the TMS Stimulator, placing the stimulus pulse in the proper location.

The DBLM uses a registration algorithm (2103) to best fit the output of the brain atlas (2101) to the exact location on the patient, utilizing the MRI data (2106). The DBLM registration algorithm (2103) scales, rotates and normalizes the image, comparing the image to the brain atlas internal image (2102). The DBLM (2100) performs a correlation between the representations, locating an offset index to be used as a correction offset between the stored brain atlas image and the patient's image. The offset, scale and rotation values are used to locate the stimulation point in the patient's data (2104).

The DBLM (2100) determines the 3 degrees of space coordinate locations of stimulus points, and outputs those locations to the ECM (1408) for stimulation. The DBLM (2100) interfaces with the ECM to allow sequencing through a set of desired stimulus application location(s).

The Brain Atlas (1407):

The Brain Atlas (1407) is preferably a component of the DBLM application (1406, 2100). The Brain Atlas (1407) includes a data base of known structural brain regions. The Brain Atlas (1407) contains multiple representations of the brain, indexed by the values dependant on entered patient data, age, size, etc. The Brain Atlas (1407) is referenced by the DBLM (1406, 2100) to establish the ideal stimulus location for a given set of outcomes by the EDMIS (1405).

The Expert Decision Making Interactive System (EDMIS) (1900):

The EDMIS (1900) is a process that includes a software module or computer application and interfaces to internal databases, offsite personnel and/or offsite databases. The EDMIS (1900) utilizes the output of the END (1902), Cognitive Test Results (1903) and input from the user (1901) to make determinations on optimal stimulus location. The EDMIS system (1900) outputs information for diagnostic purposes (1912). The EDMIS system (1900) makes determinations of the areas to be stimulated as well as treatment characteristics based on an expert diagnosis by treatment specialists (1909) and/or expert decision system (1906) using input from END (1902) and or Cognitive testing (1903), as well as trained personnel (1901).

The EDMIS (1900) utilizes patient feedback (1908) during or after the treatment session or sessions from the CSM (1412) to reassess the stimulation characteristics and instruct the CSM to modify its operation during the treatment session, by re-analyzing the data. The EDMIS (1900) allows input of results (1909) during treatment (1901), post-treatment, as well as previous output from the system, in order to reassess the patient, making suitable changes to the treatment profile, based on re-analysis by END or re-examination of Cognitive Function (1905). The EDMIS computer application or module includes a user interface (1904). The EDMIS (1900) determines the stimulation type and characteristics to be administered to the patient (1905, 1907). The EDMIS (1900) determines the type of cognitive stimulus to be used during the treatment (1907). The EDMIS (1900) interfaces to the DBLM (1911), in order to locate the exact stimulus location in a specific patient (1906), as determined by the MRI image (2106).

The Brain Co-Registry (1409):

The Brain Co-Registry (1409) may be implemented as a software module or computer application. The system may utilize an off-the-shelf Brain Co-Registry Component that implements one or more of the following functions. The Brain Co-Registry (1409) determines the region(s) of the brain to be stimulated or being stimulated by the TMS coil (1411), during the coil aiming or stimulation process. The Brain Co-Registry (1409) may assess in real-time the registration between the applied magnetic field and the stimulation location and or intensity. The Brain Co-Registry (1409) allows optimization either manually or robotically of stimulation location, relative to a pre-identified target region. The Brain Co-Registry (1409) indicates to the user the location of the brain being stimulated, using 3-D image of the brain. The Brain Co-Registry (1409) indicates the relative strength of stimulation using color-coding.

Cognitive Stimulation Module (CSM) (1412):

The CSM (1412) is a component of Embodiments A-C, and can include a computer application or a component of another application, and can be operated by a script (1413) controlled by the ECM (1408). The CSM Script (1413) can indicate the Cognitive Stimulus (1410) to be applied, the time delay between the applied Magnetic or Electrical Stimulus and the Applied cognitive stimulus (1410). The Script (1413) can include graded responses to patient feedback allowing determination of patient's progress, responses being tagged with scores for determination by the CSM of patient's progress. The CSM (1412) can apply scripted stimuli to the patient monitor, at appropriate intervals, after the ECM (1408) and TMS (1411) have applied the stimuli pulse. The CSM (1412) can accept patient feedback in the forms of answers or responses to the cognitive stimuli, making decision on treatment path in real-time.

### Magnetic Stimulator Embodiments A and B

An exemplary TMS (transcranial magnetic stimulator) (907) suitable for use in embodiments A and B is preferably FDA 51 OK approved and can be used for clinical trials, as well as deployment to treatment clinics. The TMS stimulator (907) can provide magnetic stimulus to selected regions of the brain, and allow manual placement on the head of the patient being treated (904). An appropriate fastening harness for securing to the patient's head is provided.

The location of the TMS stimulator should remain consistent during the treatment interval and should be interfaced to the ECM (905) to allow timing of the applied magnetic pulse to an accuracy of +/−5 mSec. Suitable stimulation frequencies can be of about 1 to 20 Hz for a period of about 1 to 5 seconds, with pulse envelopes lasting as long as 20 minutes for each cortical region being stimulated. The coil of the TMS Stimulator (907) should not subject the patient to temperature above 40° C. at any applied point.

### Magnetic Stimulator for Embodiment C

An exemplary Magnetic Stimulator (2503) for Embodiment C, but also usable with Embodiments A and B, is a plurality of magnetic stimulator coils (for example, 12 coils) adjustably positionable around the patient's head (2501). An integrated system combines multiple magnets and/or electrical emitters, and/or electrical chips and/or associated gyroscopes capable of detecting the precise location and vector of the electromagnetic stimulation of each electromagnetic/electrical stimulators. In addition, each electromagnetic/electrical stimulator has associated sensors capable of detecting intensity and vector of each electromagnetic/electrical stimulator, as well as electromagnetic stimulation of other electromagnetic/electrical stimulators—such that the integrated gyroscope-sensor system is capable of identifying or triangulating precise three-dimensional, single or multiple cortical or sub-cortical points in real-time.

Additional sensors can be placed at additional positions on the scalp or within intracranial orifices. In addition, a cortical or sub-cortical brain registry system allows the extrapolation/computation of the cortical or sub-cortical regions being stimulated when the electromagnetic vector(s) are applied to particular cortical or sub-cortical regions. Also, based on this integrated gyroscopic-sensor-cortical-sub-cortical registry system, real-time identification of which cortical or sub-cortical regions are being stimulated, and at what intensity, may be provided. These features allow real-time continuous adjustment and monitoring of stimulation parameters of each of the electromagnetic/electrical stimulators, until optimization of stimulation of targeted (single or multiple) cortical or sub-cortical regions has been obtained.

A system of gyroscopic components and sensors, associated with the magnetic stimulators, can continuously sense, adjust, mobilize and control the location and vector of each of the magnets or electrodes. In addition, through the use of the gyroscopic-sensor interaction vector triangulation can provide the exact position of the magnetic stimulators, and energy convergence position within a particular brain region can be identified. The intensity of each coil of a respective magnetic stimulator is controllable by the computer (1107). The Magnetic Stimulator (2503) may include a nose insertable coil, an ear insertable coil, and appropriate coils for the mouth and eyes (2501). The Magnetic Stimulator (2503) modulates the current in the coils (2501) in order to control the exact placement and intensity of the applied magnetic field, as described above, or under the direction of a commercially-available Brain Co-Registry or similar device. Large stimulator coils (2501) are capable of developing about 2 to 3 Tesla at the coils, and about 0.5 to 0.75 Tesla in the cortex at depths of up to about 5 cm. Small magnetic coils are capable of developing about 1.5 to 2 Tesla at the coils, and about 0.1 to 0.5 Tesla at depths up to about 3 to 4 cm.

The Magnetic Stimulator control system (2503) of Embodiment C controls the applied slew rate of the magnetic field, and creates magnetic field rise times from about 50 to 2000 uSec. The nose and mouth coils under the direction of the computer (2501) are able to steer and optimize the magnetic field gradient (the intensity) to deep brain areas such as the hippocampus. The stimulator coils (2501) can be mounted in a helmet or similar structure or frame placed on the patient's head (2502).

The stimulators of Embodiment C (2501) allow stimulation of single or multiple cortical or sub-cortical regions of the brain, by controlling the applied magnetic field vectors. Magnetic stimulation locations can be controlled by the computer by both control of magnetic field gradients, and robotic or inertial movement of the coils in the helmet or frame. The magnetic stimulator of Embodiment C (2503) provides magnetic field optimization through accessory coils located in the orifices of the head (2504, 2501), allowing the field to reach locations deeper and more precisely. Where appropriate, the coils can be temperature controlled.

As with the stimulator of Embodiments A and B, the magnetic stimulator of embodiment C (2503) can be provided with an interface to the ECM (1408) to allow timing of the applied magnetic pulse to an accuracy of +/−5 mSec., and allow for stimulation frequencies of about 1 to 20 Hz for a period of about 1 to 5 second, and application of pulse envelopes for a duration of up to about 30 minutes for each cortical region being stimulated.

### Electrical Stimulator for Embodiment C

The Electrical Stimulator of Embodiment C (2503) provides brain stimulation using electrical stimulation applied through a suitably located surface or invasive electrodes (2501) or magnetic or electromagnetic coils, conductors, etc. Electrical Stimulator (2503) provides precise electrode implant location details through a brain atlas derived from an MRI (1403) specific to the patient. The Electrical Stimulator (2503) can provide an interface to the ECM to allow triggered application of pulses to the patient's brain, in conjunction with applied TMS pulses or by itself. The electrical stimulator can allow the use of surface electrodes or subcutaneous electrodes, or electrodes placed and located internally or neuronally in the patient's brain.

The Electrical Stimulator (2503) can use a plurality of electrodes (for example, about 20 electrodes), supplying 10 to 100 uA stimulus pulses, controlled by the ECM (1408). Pulses can have a frequency of about 1 to 20 Hz, a pulse width of about 0.5 mSec to about 10 mSec and envelope duration of between about 10 to 200 mSec. The Electrical Stimulator (2503) should control the current applied to the stimulation electrodes, in order to place the current gradient maxima at the desired stimulation location.

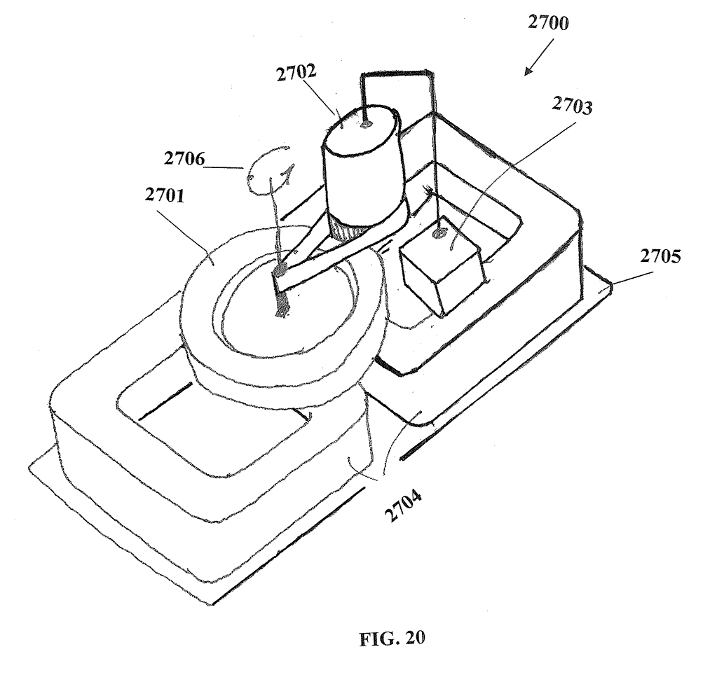

FIG. 20 schematically illustrates an exemplary embodiment of a Gyroscope Stabilization and Feedback System (2700) of the integrative neuro-cognitive system of the present invention. System (2700) includes gyroscope stabilization (2701), motor (2702) and gyroscope sensor and feedback controller (2703). System (2700) also includes at least one magnetic stimulation coil (2704) and a mounting frame (2705).

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting in any fashion. Accordingly, it is not intended that the present invention be limited to the illustrated embodiments, but only by the appended claims.

What is claimed as new and desired to be protected by Letters Patent of the United States is:

1. A neuronal medical device, comprising:
an analyzer system comprising at least one of a brain analyzer and a cognitive analyzer, the analyzer system operative to measure at least one property of an individual's condition to obtain a measured property, the measured property being related to at least one of at least one brain region and at least one cognitive feature, wherein the analyzer system also calculates a norm value from the at least one brain region or the at least one cognitive feature of healthy or diseased groups of individuals, the analyzer system comparing the measured property with the norm value and providing a resultant value; and
a stimulator system interfaced with said analyzer system including a brain stimulator module and at least one of a brain stimulator and a cognitive stimulator, where the brain stimulator includes at least one non-invasive brain stimulator operative to selectively stimulate single or multiple brain loci at least one brain region via discharge of excitatory or inhibitory stimulation energy, said stimulator module operative to position the stimulator so that stimulation energy is directed to the single or multiple brain loci at least one brain region brain region, wherein the brain region is within the left prefrontal region, frontal lobes, cingulate gyrus, hemispheres, temporal lobe, frontal lobe, parietal lobe, occipital lobe, amygdala region, cerebellum, hippocampus, anthreonal, Peabody, plaques, tangles, brain stem, medula, corpus collasum, subcortical region, cortex, gyrus, white matter and grey matter, based on the resultant value, and
the cognitive stimulator being configured to provide at least one cognitive stimulus to modify at least one cognitive feature associated with the brain region based on the resultant value.

2. A neuronal medical device, comprising:
an analyzer system comprising at least one of a brain analyzer and a cognitive analyzer operative to provide a measured property related to at least one of at least one brain region and at least one cognitive feature, calculates a norm value from the at least one brain region or the at least one cognitive feature of healthy or diseased groups of individuals, and provide a resultant comparative value; and
a stimulator system interfaced with said analyzer system and operative to selectively stimulate, at least non-invasively, single or multiple brain loci at least one brain region via discharge of excitatory or inhibitory stimulation energy, said stimulator system including a plurality of discrete non-invasive stimulators to discharge stimulation energy and positionable to direct stimulation energy at single or multiple brain loci within at least one of the left prefrontal region, frontal lobes, cingulate gyrus, hemispheres, temporal lobe, frontal lobe, parietal lobe, occipital lobe, amygdala region, cerebellum, hippocampus, anthreonal, Peabody, plaques, tangles, brain stem, medula, corpus collasum, subcortical region, cortex, gyrus, white matter and grey matter, based on the resultant value.

3. The medical device of claim 1, wherein the brain stimulator and the cognitive stimulator form a single, integrated device.

4. The medical device of claim 3, wherein the single, integrated device comprises at least one electrode in communication with at least one of ear, nose, scalp and mouth of an individual.

5. The medical device of claim 1, wherein the brain stimulator comprises at least one invasive stimulator operative to selectively provide singular or multiple site stimuli to the at least one brain region or to a different brain region.

6. The medical device of claim 1, wherein the brain stimulator is configured to provide at least one of electrical, magnetic, electromagnetic and photoelectric stimulus.

7. The medical device of claim 1, wherein the brain stimulator includes at least one electromagnetic stimulator and another source of neuronal stimulation.

8. The medical device of claim 1, further comprising a controller interfacing the analyzer system and the stimulator system to operate at least one of the brain stimulator and the cognitive stimulator.

9. The medical device of claim 1, further comprising:
a treatment module operatively connected to the analyzer system, the treatment module being configured to output data in response to the resultant value to the stimulator system; and
an in-vivo stimulator configured to subject the brain region to a treatment including at least one of cell replacement therapy, cell regenerative therapy and cell growth.

10. The medical device of claim 1, wherein the stimulators are positionable to direct stimulation energy at single or multiple brain loci in brain regions associated with Alzheimer's disease, dementia, autism spectrum disorder, mild cognitive impairment, memory loss, aging, ADHD, Parkinson's disease, depression, addiction, substance abuse, schizophrenia, bipolar disorder, memory enhancement, intelligence enhancement, concentration enhancement, well-being or mood enhancement, self-esteem enhancement, language capabilities, verbal skills, vocabulary skills, articulation skills, alterness, focus, relaxation, perceptual skills, thinking, analytical skills, executive functions, sleep enhancement, motor skills, coordination skills, sports skills, musical skills, inter-personal skills, social skills and affective skills.

11. The medical device of claim 10, wherein the stimulators are positionable to direct stimulation energy at single or multiple brain loci in brain regions associated with Alzheimer's disease.

12. A medical device for treatment of a brain-related condition or for enhancement of a cognitive function in an individual, comprising:
a stimulator system comprising:
at least one brain stimulator configured to at least selectively and non-invasively stimulate a localized brain region, wherein the localized brain region is associated with the brain-related condition or with the cognitive function; and at least one cognitive stimulator configured to selectively stimulate at least one cognitive feature associated with the localized brain region; and
an in-vivo stimulator configured to subject the localized brain region to a treatment including at least one of cell replacement therapy, cell regenerative therapy and cell growth.

13. The medical device of claim 12, further comprising:
an analyzer system, including at least one of a brain analyzer and a cognitive analyzer, the analyzer system being operative to measure at least one property of the brain related condition to obtain a measured property, the measured property being related to at least one of at least one brain region and at least one cognitive feature, wherein the analyzer system also calculates a norm value from at least one brain region or at least one cognitive feature of healthy or diseased groups of individuals, the analyzer system comparing the measured property with the norm value and provide a resultant value to the stimulator system; and
a controller interfacing the analyzer system and the stimulator system to operate at least one of the brain stimulator and the cognitive stimulator.

14. The medical device of claim 12, wherein the brain stimulator system is a helmet.

15. A medical device comprising:
an analyzer system comprising at least one of a brain analyzer and a cognitive analyzer, the analyzer system being operative to measure at least one property of an individual's condition to obtain a measured property, the measured property being related to at least one of at least one brain region and at least one cognitive feature, wherein the analyzer system also calculates a norm value from the at least one brain region or the at least one cognitive feature of healthy or diseased groups of individuals, the analyzer system comparing the measured property with the norm value and providing a resultant value;
a treatment module operatively connected to the analyzer system, the treatment module being configured to output data in response to the resultant value;
a first stimulator operative to discharge excitatory or inhibitory stimulation and configured to receive data from the treatment module and position energy discharging stimulators to stimulate the at least one brain region; and
a second stimulator associated with the first stimulator, the second stimulator being configured to activate the at least one cognitive feature of the individual.

16. The medical device of claim 15, wherein the first stimulator comprises at least one non-invasive brain stimulator and at least one invasive brain stimulator, wherein the at least one non-invasive brain stimulator and the at least one invasive brain stimulator are configured to selectively stimulate the at least one brain region.

17. The medical device of claim 15, wherein the non-invasive brain stimulator comprises at least one of electrical, magnetic, electromagnetic and photoelectric electrodes.

18. The medical device of claim 15, wherein the first stimulator comprises at least one invasive brain stimulator configured to selectively stimulate the at least one brain region.

19. The medical device of claim 15, wherein the second stimulator is configured to provide at least one cognitive stimulus relevant to the cognitive feature.

20. The medical device of claim 15, wherein the first stimulator provides non-invasive stimulation energy.

21. The medical device of claim 15, further comprising an assessment device configured to provide feedback to at least one of the first stimulator and the second stimulator, based on responses to stimuli from the first and second stimulators.

22. The medical device of claim 15, further comprising a controller configured to operate the first and second stimulators in response to the resultant value.

23. A method of treating cognitive impairments, comprising the steps of:
identifying a brain region associated with a cognitive impairment;
subjecting the brain region to at least one of electrical, magnetic, electromagnetic and photoelectric stimuli; and
simultaneously, modifying a cognitive function associated with the brain region.

24. The method of claim 23, further comprising the steps of:
subjecting the brain region to a treatment including at least one of cell replacement therapy, cell regenerative therapy and cell growth; and
optionally, subjecting the brain region to a pharmacological treatment.

25. A method of therapy for impaired cognitive functions, the method comprising the steps of:
providing a first stimulus to a pre-defined brain region of an individual without stimulating physically adjacent brain regions, the pre-defined brain region being functionally associated with an impaired cognitive function; and
providing at least one cognitive stimulus to the patient, to elicit a response involving the impaired cognitive function.

26. The method of claim 25, further comprising the steps of:
measuring at least one local brain function of the pre-defined brain region to obtain a local measurement;
comparing the local measurement to a normative value to obtain an evaluation data; and
adjusting, in response to the evaluation data, at least one of the steps of providing a first stimulus and providing at least one cognitive stimulus.

27. The method of claim 25, further comprising:
measuring the response to the first stimulus and to the cognitive stimulus, to obtain a cognition data; and
adjusting, based on the cognition data, at least one of the steps of providing a first stimulus and providing at least one cognitive stimulus.

28. A method of brain therapy, comprising:
providing an analyzer system including at least one of a brain analyzer and a cognitive analyzer;
the analyzer system measuring at least one property of an individual's condition to obtain a measured property related to at least one of at least one brain region and at least one cognitive feature;
the analyzer system calculating a norm value from at least one brain region or at least one cognitive feature of healthy or diseased groups of individuals; and
the analyzer system comparing the measured property value with the norm value to provide a resultant value.

29. A method of therapy, comprising:
measuring a local brain function of at least one brain region to produce a local measurement relative to a norm value of an individual;
evaluating the local measurement relative to a normative value of a group of individuals, to produce an evaluation datum;
adjusting at least one of one or more non-invasive stimulators and one or more one cognitive stimulators in response to the evaluation datum; and
providing stimuli from the at least one or more non-invasive stimulators and the least one or more cognitive stimulators to the at least one brain region.

30. The method of claim 29, further comprising:
measuring a response to the stimuli to produce a cognition measurement; and
adjusting at least one of the non-invasive and cognitive stimulators in response to the cognition measurement.

31. The method of claim 29, wherein the step of providing non-invasive stimuli includes providing electromagnetic stimuli and another form of neuronal stimuli, to selectively stimulate the at least one brain region.

32. A method of therapy for a cognitive symptom of a disease, comprising:
selectively stimulating at least one brain region functionally associated with a cognitive symptom, without physically stimulating adjacent regions; and
providing at least one cognitive stimulus selected to elicit a response involving the cognitive symptom.

33. The method of claim 32, further comprising:
measuring local brain function at the at least one brain region to produce a local measurement;
evaluating the local measurement relative to a normative value to produce an evaluation datum; and
adjusting at least one of the brain region stimulating and the at least one cognitive stimulus in response to the evaluation datum.

34. The method of claim 32, further comprising:
measuring the response to the stimulus to produce a cognition measurement; and
adjusting at least one of the selective stimulating and the at least one cognitive stimulus in response to the cognition measurement.

35. The method of claim 32, wherein the at least one brain region is a brain deficient region.

36. The method of claim 32, wherein the at least one brain region is associated with a memory function disease.

37. The method of claim 32, wherein the at least one brain region is associated with Alzheimer's disease.

38. A method of neuronal therapy comprising:
providing an analyzer system including at least one of a brain analyzer and a cognitive analyzer;
the analyzer measuring at least one property of an individual's condition, the measured property being related to at least one of at least one brain region and at least one cognitive feature;
the analyzer calculating a norm value from at least one brain region or at least one cognitive feature of healthy or diseased groups of individuals;
the analyzer comparing the measured property with the norm value and provide a resultant value;
providing a stimulator system including:
providing one or more non-invasive stimulators to selectively provide singular or multiple site stimuli to at least one brain region associated with the property;
providing one or more cognitive stimulators to selectively provide cognitive stimuli to at least one cognitive function relevant to the at least one property; and
providing a controller and interfacing the controller with the analyzer system and the stimulator system, the controller operating at least one of the one or more non-invasive brain stimulators and one or more one cognitive stimulators.

39. A method of neuronal therapy, comprising:
providing a module with a plurality of discrete energy emitting stimulators;
positioning the module on a patient;
positioning said discrete stimulator to direct stimulation energy at single or multiple brain loci in brain regions associated with Alzheimer's disease, dementia, autism spectrum disorder, mild cognitive impairment, memory loss, aging, ADHD, Parkinson's disease, depression, addiction, substance abuse, schizophrenia, bipolar disorder, memory enhancement, intelligence enhancement, concentration enhancement, well-being or mood enhancement, self-esteem enhancement, language capabilities, verbal skills, vocabulary skills, articulation skills, alertness, focus, relaxation, perceptual skills, thinking, analytical skills, executive functions, sleep enhancement, motor skills, coordination skills, sports skills, musical skills, inter-personal skills, social skills and affective skills; and
stimulating said associated single or multiple brain loci.

40. A system for neuronal assessment and treatment, comprising:
a computer and optional control module;
a user energy stimulation module optionally including a TMS stimulator;
a cognitive stimulation module;
a diseased brain localization module; and
an optional cognitive testing module.

41. The system of claim 40 further including a cognitive progress monitoring module.

42. The system of claim 40, further including an associated comparative norm data base module interfaced with said computer.

43. A medical treatment device comprising,
a computer system with user access;
synchronized magnetic and cognitive training stimulator systems for applying a stimulus;
an Executive Control Module for managing the sequencing and state of the treatment session and application of stimulus;
a decision making system based on patient response making determinations based on that response, alerting an operator or modifying script to optimize cognitive training; and
and END module for determining the presence of Alzheimer's disease.
